(19) United States
(12) Patent Application Publication
(10) Pub. No.: US 2019/0133980 A1
(43) Pub. Date: May 9, 2019

(54) Compositions and methods for targeting and treating homologous recombination-deficient tumors

(71) Applicant: YALE UNIVERSITY, New Haven, CT (US)

(72) Inventors: Ranjit BINDRA, New Haven, CT (US); Peter GLAZER, Guilford, CT (US); Nathaniel ROBINSON, Philadelphia, PA (US); Parker SULKOWSKI, Guilford, CT (US); Christopher CORSO, East Haven, CT (US)

(21) Appl. No.: 16/306,215

(22) PCT Filed: Jun. 2, 2017
(86) PCT No.: PCT/US17/35756

§ 371 Date: Nov. 30, 2018

Related U.S. Application Data
(60) Provisional application No. 62/344,678, filed on Jun. 2, 2016.
(60) Provisional application No. 62/451,122, filed on Jan. 27, 2017.

(51) Int. Cl. A61K 31/194; A61K 45/06; A61P 35/00; A61K 31/502; A61K 31/166; A61K 31/454; A61K 31/4184; A61K 31/55; A61K 31/5025

(52) U.S. Cl. CPC A61K 31/194; A61K 45/06; A61P 35/00; A61K 31/502; A61K 31/5025; A61K 31/454; A61K 31/4184; A61K 31/55; A61K 31/166

(57) ABSTRACT

The invention includes compositions and methods for treating or preventing a cancer in a subject. In one aspect the invention provides methods of administering to a subject suffering from a cancer with cells containing an IDH1 or IDH2 mutation, at least one compound comprising a DNA repair inhibitor. The invention also provides methods of treating a cancer with 2 HG, a derivative of 2-HG, any variant and any mixtures thereof. In one aspect the invention provides a pharmaceutical composition comprising an anti-tumor effective mount of at least one compound selected from the group consisting of 2-hydroxyglutarate (2-HG), a derivative of 2-HG, any variant and any mixtures thereof.

### CROSS-REFERENCE TO RELATED APPLICATIONS

The present application claims priority under 35 U.S.C. § 119(e) to U.S. Provisional Patent Application No. 62/344,678, filed Jun. 2, 2016 and U.S. Provisional Patent Application No. 62/451,122, filed Jan. 27, 2017, which are incorporated herein by reference in their entirety.

### STATEMENT REGARDING FEDERALLY SPONSORED RESEARCH OR DEVELOPMENT

This invention was made with government support under grants R01CA168733, R01CA177719 and R01ES005775 awarded by National Institutes of Health. The government has certain rights in the invention.

### BACKGROUND OF THE INVENTION

Genomic instability is a hallmark of cancer cells. To maintain genomic stability and ensure high-fidelity transmission of genetic information, cells have evolved a complex mechanism to repair DNA double-strand breaks (DSBs), through homologous recombination (HR). Frequently, the inability to properly coordinate repair of damaged DNA underlies tumorigenesis and disease progression in malignancies. Many human cancer syndromes have been linked to mutations in DNA repair pathway. For instance, germline mutations in the tumor suppressors BRCA1 and BRCA2, two critical HR repair mediators, predispose to both breast and ovarian cancer. However, HR-mediated DNA repair deficiency also sensitizes cancer cells to DNA-damage-inducing therapy such as radiation therapy and DNA-damage-based chemotherapy. One of the most exciting recent therapeutic breakthroughs in cancer is identification of a synthetic lethal interaction between HR repair deficiency and poly(ADP-ribose) polymerase (PARP) inhibition. PARP inhibitors inhibit single-strand DNA repair, which leads to DSBs when DNA replication occurs. Normal cells can repair these DSBs. However, HR repair-deficient cancer cells cannot repair PARP-inhibitor-induced DSBs and die when treated with these drugs. Thus, PARP inhibitors can selectively target HR repair-deficient cancer.

The normal function of the metabolic enzyme isocitrate dehydrogenase-1 (IDH1) is to catalyze the conversion of isocitrate to α-ketoglutarate (α-KG) in the citric acid cycle. Recurring mutations in IDH1, and its mitochondrial homolog isocitrate dehydrogenase-2 (IDH2), were identified in hematologic and solid tumor malignancies, including gliomas and acute myeloid leukemia (AML) where mutations occur in more than 70% of low grade gliomas and up to 20% of higher grade tumors (e.g., secondary glioblastoma multiforme; GBM), and approximately 10% of AML cases. When mutated, IDH1 and IDH2 acquire a neomorphic function and convert isocitrate to 2-hydroxyglutarate (2-HG) instead of the normal product, α-KG. This alters the cells' genetic programming, and instead of maturing, the cells remain primitive and proliferate quickly. Emerging research indicates that 2-HG is an oncometabolite, with pleiotropic effects on cell biology including chromatin methylation and cellular differentiation, although many questions remain unclear about its impact on tumorigenesis and therapy response. Currently, with the aim of blocking 2-HG production, IDH1 and IDH2 inhibitors are being developed and tested in clinical trials for both glioma and AML, with the underlying assumption that blocking IDH neomorphic activity alone will reduce or eliminate tumor growth.

There is a need in the art to develop novel therapeutics which specifically target and exploit the molecular mechanisms related to IDH defects and 2-HG production. There is also a need to identify and develop novel compositions that inhibit DNA repair to render tumor cells sensitive to PARP inhibition. The present invention addresses and meets these needs.

### SUMMARY OF THE INVENTION

In one aspect the invention comprises a method of treating or preventing a cancer in a mammalian subject in need thereof, the method comprising administering to the subject a therapeutically effective amount of at least one compound wherein the compound is selected from the group consisting of a DNA repair inhibitor, a DNA strand break repair inhibitor, and a homologous recombination (HR) repair inhibitor, wherein cells in the cancer comprise an isocitrate dehydrogenase (IDH) mutation.

In various embodiments, the IDH mutation comprises a mutation in IDH1 or IDH2.

In various embodiments, the at least one compound comprises at least one poly(ADP-ribose) polymerase (PARP) inhibitor selected from the group consisting of olaparib, Iniparib, Niraparib, Veliparib, Rucaparib, 3-aminobenzamide and BMN-673 (Talazoparib), or at least one alpha-ketoglutarate-dependent dioxygenase A or B (KDM4A or KDM4B) inhibitor selected from the group consisting of DMOG, NSC 636819, PK 118 310, NCGC 00247751, NCGC 00244536, NCGC 00247743, IXO1, Disulfiram and JIB04.

In various embodiments, the subject is further administered at least one antitumor agent.

In various embodiments, the antitumor agent is selected from the group consisting of a topoisomerase inhibitor, an alkylating agent, nitrosoureas, an antimetabolite, an antitumor antibiotic, an antimicrotubule agent, a hormonal agent, a DNA strand break inducing agent, an epidermal growth factor (EGF) receptor inhibitor, an anti-EGF receptor antibody, an AKT inhibitor, an mTOR inhibitor, a CDK inhibitor, a tyrosine kinase receptor

(TKR) inhibitor, a serine/threonine kinase inhibitor, a phosphatidyl inositol 3-kinase-like (PIKK) protein kinase inhibitor, a DNA dependent protein kinase (DNA-PK) inhibitor, an Ataxia Telangiectasia Mutated (ATM) inhibitor, an Ataxia Telangiectasia and Rad3 Related (ATR) inhibitor, a ribonucleotide reductase inhibitor, and an immune checkpoint inhibitor. In various embodiments, treatment of the subject with the at least one compound and at least one antitumor agent is synergistic.

In various embodiments, the at least one compound and at least one antitumor agent are co-administered to the subject.

In various embodiments, the at least one compound and at least one antitumor agent are coformulated for administration to the subject.

In various embodiments, the subject is further administered radiation therapy.

In various embodiments, the treatment of the subject with at least one compound and the radiation therapy is synergistic.

In various embodiments, the at least one compound is administered to the subject by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, pleural, peritoneal, subcutaneous, epidural, otic, intraocular, and topical.

In various embodiments, the cancer comprises at least one selected from the group consisting of brain head and neck cancer, glioma, meningioma, glioblastoma multiforme, lymphoma, leukemia, acute myeloid leukemia (AML), cholangiocarcinoma, multiple myeloma and neuroblastoma.

In various embodiments, the cancer comprises glioma, acute myelogenous leukemia or cholangiocarcinoma.

In various embodiments, the mammal is a human.

In another aspect the invention comprises a method of treating a cancer in a mammalian subject in need thereof, the method comprising administering to the subject a therapeutically effective amount of at least one compound selected from the group consisting of 2-hydroxyglutarate (2-HG), a derivative of 2-HG, any variant and any mixtures thereof.

In various embodiments, the cancer does not comprise an isocitrate dehydrogenase (IDH) mutation.

In various embodiments, the at least one compound induces a defect in DNA repair, DNA strand break repair or a homologous recombination (HR) of the cancer cells in the subject.

In various embodiments, the subject is further administered at least one antitumor agent and one poly(ADP-ribose) polymerase (PARP) inhibitor.

In various embodiments, the PARP inhibitor is at least one selected from the group consisting of olaparib, Iniparib, Niraparib, Veliparib, Rucaparib, 3-aminobenzamide and BMN-673 (Talazoparib).

In various embodiments, the antitumor agent is at least one selected from the group consisting of a topoisomerase inhibitor, an alkylating agent, nitrosoureas, an antimetabolite, an antitumor antibiotic, an antimicrotubule agent, a hormonal agent, a DNA strand break inducing agent, an epidermal growth factor (EGF) receptor inhibitor, an anti-EGF receptor antibody, an AKT inhibitor, an mTOR inhibitor, a CDK inhibitor, a tyrosine kinase receptor (TKR) inhibitor, a serine/threonine kinase inhibitor, a PIKK protein kinase inhibitor, a DNA-PK inhibitor, an ATM inhibitor, an ATR inhibitor, a ribonucleotide reductase inhibitor, and an immune checkpoint inhibitor.

In various embodiments, the treatment of the subject with the at least one compound, the at least one antitumor agent and the PARP inhibitor is synergistic.

In various embodiments, the at least one compound, the at least one additional antitumor agent and the PARP inhibitor are co-administered to the subject.

In various embodiments, the at least one compound, the at least one additional antitumor agent and the PARP inhibitor are coformulated for administration to the subject.

In various embodiments, a PARP inhibitor and a radiation therapy are further administered to the subject.

In various embodiments, treatment of the subject with the at least one compound and the PARP inhibitor and radiation therapy is synergistic.

In various embodiments, the at least one compound is administered to the subject by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, pleural, peritoneal, subcutaneous, epidural, otic, intraocular, and topical.

In various embodiments, the cancer comprises at least one selected from the group consisting of breast cancer, prostate cancer, ovarian cancer, cervical cancer, skin cancer, pancreatic cancer, colorectal cancer, renal cancer, liver cancer, brain cancer, glioma, meningioma, glioblastoma multiforme, melanoma, lymphoma, leukemia, acute myeloid leukemia (AML), cholangiocarcinoma, lung cancer, endometrial cancer, head and neck cancer, sarcoma, multiple myeloma and neuroblastoma.

In various embodiments, the cancer comprises cells defective in at least one protein selected from the group consisting of BRCA1, BRCA2, PTEN, ATM, ATR, PALB2, FANCD2, RAD50, RAD51, other component of the homology dependent DNA repair pathway or the non-homologous end joining pathway or other component that mediate or regulate DNA repair.

In various embodiments, the derivative of 2-HG is (2R)-octyl-α-hydroxyglutarate (octyl-(R)-2HG).

In various embodiments, the mammal is a human.

In another aspect the invention comprises a pharmaceutical composition comprising an anti-tumor effective amount of at least one compound selected from the group consisting of 2-HG, a derivative of 2-HG, any variant thereof, and any mixtures thereof.

### BRIEF DESCRIPTION OF THE DRAWINGS

The following detailed description of specific embodiments of the invention will be better understood when read in conjunction with the appended drawings. For the purpose of illustrating the invention, there are shown in the drawings specific embodiments. It should be understood, however, that the invention is not limited to the precise arrangements and instrumentalities of the embodiments shown in the drawings.

FIGS. 1A-1M are a series of images, graphs and histograms illustrating the finding that mutant IDH1 suppresses homologous recombination via 2-hydroxyglutarate. FIG. 1A, CRISPR/Cas-9 targeting strategy for generating a heterozygous IDH1-R132H mutant astrocyte clone. FIGS. 1B-1C, Sanger sequencing and Western blot confirm that this clone contains a mutation at the IDH1 locus that results in expression of the mutant enzyme. FIG. 1D, An enzyme-based assay demonstrates the mutant cells secrete high levels of 2HG. FIG. 1E, Clonogenic survival assay in wild-type or IDH1 mutant astrocytes in response to ionized radiation (IR). FIG. 1F, Representative images and FIG. 1G, quantification of neutral comet assay 24-hr post 5 Gy IR in wild-type and IDH1-R132H heterozygous astrocytes (N=3 biological replicates with >50 cells per replicate). FIG. 1H, Host cell reactivation assays specific for HR and NHEJ performed in wild-type or IDH1-R132H heterozygous astrocytes. FIG. 11, U2OS-DRGFP chromosomal HR reporter assay performed after 10 days of culture in 300 μM octyl-(R)-2-hydroxyglutarate or DMSO control. Note the suppression of HR is comparable in magnitude to RAD51 or BRCA2 suppression by siRNA knockdown. FIG. 1J, Analysis of BRCA1, FANCD2, and RAD51 expression in embryonic brain, IDH1 wild-type or IDH1-R132H glioma patient samples. FIG. 1K, mRNA level of indicated HR and NHEJ factors determined by RNA-sequencing from low-grade glioma patients comparing IDH1 wild-type (N=64) and IDH1-R132H (N=203) samples. FIG. 1L, Relative BRCA1, FANCD2, RAD51 mRNA levels in wild-type or IDH1-R132H heterozygous astrocytes normalized to wild-type. FIG. 1M, Western blot analysis of BRCA1, FANCD2, RAD51 protein levels in wild-type or IDH1-R132H heterozygous astrocytes with quantification normalized to Vinculin.

FIGS. 2A-2I are a series of graphs, histograms and drawings depicting the synthetic lethal interaction between IDH1 mutations and PARP inhibition. FIG. 2A, Short-term growth delay assay in wild-type or IDH1-R132H heterozygous astrocytes treated with indicated amounts of the PARP inhibitor BMN-673. Note four-fold decrease in IC₅₀ in R132H mutants. FIG. 2B, Long-term colony forming assay confirms differential sensitivity to BMN-673. Note ten-fold increase in sensitivity at 10 nM BMN-673. FIG. 2C, Short-term growth delay assay in wild-type HCT116 cells following 4-day culture in indicated concentration of octyl-(R)-2-hydroxyglutarate or DMSO control, either treated with 300 nM BMN-673 or DMSO. Note that fractional survival is normalized to survival in either DMSO or 300 nM BMN-673 without octyl-(R)-2HG. FIG. 2D, Same as FIG. 2C, but dose-response to BMN-673 after treatment with either 900 μM octyl-(R)-2HG or DMSO. FIG. 2E, Synergy surface plot comparing wild-type and IDH1-R132H heterozygous astrocytes treated with indicated doses of BMN-673 and Cisplatin. Darker shading indicates increased synergy. FIG. 2F, Bar graph highlighting IDH1-R132H-dependent synergy between 8 nM BMN-673 and 1 cisplatin. Note difference between IDH1 wild-type and IDH1-R132H mutant astrocytes treated with both BMN-673 and cisplatin. FIG. 2G, Absolute tumor volumes of subcutaneous flank xenografts of either wild-type or IDH1-R132H heterozygous HCT116 cells after treatment with BMN-673 versus vehicle alone. FIG. 2H, Kaplan-Meyer survival curve comparing either wild-type or IDH1-R132H heterozygous HCT116 xenografts with BMN-673 treatment, using 3-fold tumor growth as an endpoint. Note significant difference. FIG. 21, Proposed mechanism by which the (R)-2-hydroxyglutarate produced by IDH1-R132H induces an HR-deficient “BRCAness” phenotype and subsequent vulnerability to PARP inhibition.

FIGS. 3A-3I are a series of images, graphs and histograms illustrating the preparation of IDH1 mutant. FIG. 3A, T7 endonuclease assay confirms guide RNA-directed cleavage of Cas9 at the IDH1 locus. FIG. 3B, TOPO cloning and subsequent Sanger sequencing of clones from putative IDH1 R132H heterozygous astrocyte genomic DNA demonstrates heterozygous introduction of the IDH1-R132H mutation. FIG. 3C, RFLP analysis of wild-type or single cell-selected astrocyte clone in the presence of either mock or Bcl1 restriction endonuclease. FIG. 3D, Short-term growth of wild-type or IDH1-R132H heterozygous astrocytes co-incubated with the indicated concentration of the mutant IDH1-specific small molecule inhibitor AGI-5198 or DMSO control. Emission measures relative concentration of 2HG in the conditioned media. FIG. 3E, Enzyme-based assay for intracellular concentration of NAD+ in wild-type or IDH1-R132H heterozygous astrocytes per 1M cells. FIG. 3F, Short-term growth delay assay of WT and IDH1-R132H heterozygous astrocytes. FIG. 3G, Schematic representation of host-cell reactivation assays specific for HR or NHEJ. FIG. 3H, Representative images and FIG. 3I, quantification of neutral comet assay on wild-type or IDH1-R132H heterozygous astrocytes at baseline (no IR). Wild-type cells are cultured for 10 days in either 300 μM octyl-(R)-2HG or DMSO

FIGS. 4A-4C are a series of images, graphs and histograms depicting short-term cell growth inhibition assay with IDH1-WT and -mutant cells profiled against small molecule DNA damage response and DSB repair inhibitors. FIG. 4A, Schematic of short-term growth delay assay workflow for high-throughput drug screening. FIG. 4B,

Representative positive control of high-throughput short-term growth delay assay on DLD1 wild-type or BRCA-null cells in the presence of the indicated concentration of the PARP inhibitor olaparib. FIG. 4C, Short-term growth delay assays on wild-type or IDH1-R132H heterozygous astrocytes treated with the indicated concentration of selected DNA repair inhibitors.

FIGS. 5A-5F are a series of images, graphs and histograms illustrating the finding that other cell lines are sensitive to PARP inhibitors. FIG. 5A, Western blot of wild-type and IDH1-R132H heterozygous HCT116 cells and glioma cell lines LN18 and LN229 for IDH1 and IDH1-R132H, with SMC1 as loading control. FIG. 5B, enzyme-based assay for secreted 2HG on conditioned media from wild-type or IDH1-R132H heterozygous HCT116 cells normalized to wild-type astrocytes (not shown). FIG. 5C, Neutral comet assay with representative images on wild-type or IDH1-R132H heterozygous HCT116 cells. PK=DNA-PK null HCT116 cells, which serve as positive control. FIG. 5D, Short-term growth delay assay on wild-type and IDH1-R132H heterozygous HCT116 cells with the indicated concentration of BMN-673. Note nearly eight-fold difference in IC₅₀ between cell lines. FIG. 5E, Long-term colony forming assay on wild-type or IDH1-R132H heterozygous HCT116 cells with the indicated concentration of BMN-673. Note roughly ten-fold difference in cell viability at 10 nM BMN-673. FIG. 5F, Short-term growth delay assay on wild-type or IDH1-R132H heterozygous astrocytes with a selected panel of PARP inhibitors at the given concentrations.

FIGS. 6A-6E are a series of images, graphs and histograms depicting that IDH1-WT and -mutant cells also exhibit sensitization to cisplatin, MMS, and irinotecan as single DNA damaging agents. FIG. 6A, Representative short-term growth delay assays on wild-type or IDH1-R132H heterozygous astrocytes with the indicated concentration of selected DNA damaging agents. FIG. 6B, Short-term growth delay assay on wild-type or IDH1-R132H heterozygous astrocytes either with or without 1 μM cisplatin (CDDP), with the indicated concentration of BMN-673. FIG. 6C, Matrix representation of short-term growth delay assay on IDH1-R132H heterozygous HCT116 with the indicated concentration of BMN-673 or TMZ. Darker shading indicates increased synergistic interaction between the two drugs. FIG. 6D, Treatment schematic of mouse tumor cell injection and treatment schedule with BMN-673. FIG. 6E, Median tumor volume of wild-type or IDH1-R132H heterozygous HCT116 flank xenografts. Treatment with BMN-673 was started on day 16 (arrow).

FIGS. 7A-7H are a series of images, graphs, histograms, and a table showing that 2-Hydroxyglutarate is sufficient to induce homologous recombination deficiency and PARP inhibitor synthetic lethality. FIG. 7A, Quantification of neutral comet assay performed in WT U2OS cells are 24 h exposure to indicated amounts of the cell permeable, a-ketoglutarate-dependent dioxygenase inhibitor, DMOG. FIG. 7B, Quantification of U2OS DR-GFP assay performed after 24 h pretreatment with indicated concentrations of DMOG or a-ketoglutarate. FIG. 7C, Robust Z-Score of normalized HR from the ligand-inducible DR-GFP of 64 smart pool siRNAS targeting a-ketoglutarate dependent dioxygenases compared to the effects of 41 1 mM (R)-2HG, (S)-2HG and DMOG, as well as positive control siRNAs targeting known core DNA repair proteins, and negative control siRNAs: Non targeting pool siRNAS, siSCR, siRISC, siGAPDH and mock siRNA transfection. FIGS. 7D-7E, Quantitation of (FIG. 7D) ligand inducible DR-GFP and (FIG. 7E) neutral comet assay with deconvoluted siRNAs against KDM4A and KDM4B each performed 96 h after siRNA transfection in U2OS inducible DR-GFP cells. FIG. 7F, Quantification and representative images of neutral comet assay performed in WT and R132H/+HCT116 cells treated with the KDM4 inhibitor NSC 636819. FIG. 7G, Cell cycle phase distribution by DNA content in DMSO or DMOG treated cells. FIG. 7H, List of siRNAs targeting Alpha-ketoglutarate dependent dioxygenases and core DNA repair genes.

FIGS. 8A-8G are a series of a table, images, graphs and histograms illustrating IDH1 mutation- and 2HG-dependent DSB repair defects in patient-derived primary acute myeloid leukemia samples. FIG. 8A, Patient information and clinical characteristics for the matched pair primary AML samples. FIG. 8B, Cell cycle analysis of the IDH1 WT and R132H primary AML samples. FIG. 8C, Quantification and FIG. 8D, representative images of neutral comet assays performed on primary AML patient cells. FIG. 8E, Radiation survival of primary AML cultures assayed by a short term viability assay 48 h after 5 Gy IR. FIG. 8F, Representative images of neutral comet assay performed 24 h post 5 Gy IR on the Primary AML cells WT-1 and Mut-1(IDH1 R132H) indicating differential persistence of DNA DSBs post IR. FIG. 8G, Proposed mechanism by which the (R)-2-hydroxyglutarate produced by IDH1 R132H (or (S)-2HG produced by hypoxia) induces an HR-deficient “BRCAness” phenotype and subsequent vulnerability to PARP inhibition.

### DETAILED DESCRIPTION OF THE INVENTION

The present invention relates in part to the unexpected discovery of novel compounds that sensitize a tumor cell to anticancer therapies that comprise at least one of 2-hydroxyglutarate (2-HG), a derivative of 2-HG, and any variant or mixtures thereof. The present invention also includes methods for treating or preventing cancer in a subject, wherein the cancer cells have an IDH1 or IDH2 mutation.

Radiation therapy and chemotherapy are frequently used in cancer treatment, but unfortunately innate or acquired resistance to these therapies remains a major clinical challenge in oncology. The development of compounds that sensitize tumor cells to established therapies thus represents an attractive approach to optimize therapy and extend survival and quality of life in patients. As demonstrated herein, the present invention provides a novel class of DNA double-strand break repair inhibitors that exhibits potent synthetic lethal activity in the setting of DNA damage response and DNA repair defects.

As demonstrated herein, treatment with the compound of the invention is synergistic with hypoxia and PARP inhibition, and this synergism is amplified further in the context of a cancer with IDH1 or IDH2 mutation. Without wishing to be limited by any theory, the mechanism of action of this class of 2-HG compounds, derivatives and variants thereof appears to be related to inhibition of homologous recombination repair.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

As used herein, the articles “a” and “an” are used to refer to one or to more than one (i.e., to at least one) of the grammatical object of the article. By way of example, “an element” means one element or more than one element.

As used herein, “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “antitumor agent” or “chemotherapeutic agent” refers to a compound or composition that may be used to treat or prevent cancer. Non-limiting examples of these agents are DNA damaging agents, such as topoisomerase inhibitors (for example, etoposide, camptothecin, topotecan, irrinotecan, teniposide, mitoxantrone), antimicrotubule agents (for example, vincristine, vinblastine and taxanes), antimetabolite agents (for example, cytarabine, methotrexate, hydroxyurea, 5-fluorouracil, flouridine, 6-thioguanine, 6-mercaptompurine, fludaribine, pentostatin, cholorodeoxyadenosine), DNA alkylating agents (for example, cisplatin, mecholorethamine, cyclophosphamide, ifosphamide, melphalan, chlorumbucil, busulfan, thiotepa, carmustine, lomustine, carboplatin, dacarbazine, procarbazine, temozolomide) and DNA strand break inducing agents (for example, bleomycin, doxarubicin, daunorubicine, idarubicine, mitomycin C).

Antitumor agents include but are not limited to avicin, aclarubicin, acodazole, acronine, adozelesin, adriamycin, aldesleukin, alitretnoin, allopurinl sodium, altretamine, ambomycin, amitantrone acetate, aminoglutethimide, amscrine, anastrazole, annoceous acetogenins, anthramycin, asimicin, asparaginase, asperlin, azacitidine, azetepa, azotomycin, batimstat, benzodepa, bexarotene, bicalutamide, bisantrene, bisanafide, bizelesin, bleomycin, brequinar, brompirimine, bullatacin, busulfan, cabergoline, cactinomycin, calusterone, caracemide, carbetimer, carbopltin, carmustine, carubicin, carzelesin, cedefingol, chlorumbucil, celecoxib, cirolemycin, cisplatin, cladiribine, crisnatol, cyclophosphamide, cytarabine, dacarbazine, DACA, dactinomycin, daunorubicin, daunomycin, decitabine, denileukine, dexormaplatin, dezaguanine, diaziqone, docetaxel, doxarubicin, droloxifene, dromostalone, duazomycin, edatrexate, eflornithin, elsamitrucin, estramustine, etanidazole, etoposide, etropine, fadrozole, fazarabine, feneretinide, floxuridine, fludarabine, flurouracil, fluorocitabine, 5-FdUMP, fosquidone, fosteuecine, FK-317, FK-973, FR-66979, FR-900482, gemcitabine, gemtuzumab, ozogamicin, Gold Au198, goserelin, guanacone, hydroxyurea, idarubicine, ilmofosine, interferon alpha and analogs, iprolatin, irinotecan, lanreotide, letrozole, leuprolide, liarozole, lometrexol, lomustine, losoxantrone, masoprocol, maytansine, maturedepa, mecholoroethamine, megesterol, melengesterol, melphalan, menogaril, metoprine, mycophenolic acid, mitindomide, mitocarcin, mitogillin, mitomalacin, mitomycin, mitomycin C, mitosper, mitotane, mitoxantrone, nocodazole, nogalamycin, oprelvekin, ormaplatin, profiromycin, oxisuran, paclitaxel, pamidronate, pegaspargase, peliomycin, pentamustin, peplomycin, perfosfamide, pipobroman, piposulfan, piroxantrone, plicamycin, plomestane, porfimer, prednimustin, procarbazine, puromycin, pyrazofurin, riboprine, rogletimide, rituximab, rolliniastatin, safingol, samarium, semustine, simtrazene, sparfosate, sparcomycin, sulphofenur, spirogermanium, spiromustin, spiroplatin, squamocin, squamotacin, streptozocin, streptonigrin, SrCl₂, talosmycin, taxane, taxoid, tecoglan, temoprofin, tegafur, teloxantrone, teniposide, terxirone, testolactone, thiamiprine, thiotepa, thymitaq, tomudex, tiazofurin, tirapamazine, Top-53, topetecan, toremixifine, trastuzumab, trestolone, tricribine, trimetrexate, tricribine, trimetrexate glucuronate, triptorelin, tubulozole, uracil mustard, valrubicine, uredepa, vapreotide, vinblastin, vincristine, vindesin, vinepidine, zinostatin, vinglycinate, vinleurosine, vinorelbine, vinrosidine, vinzolidine, vorozole, zeniplatin, zorubicine, 2-chlorodeoxyrubicine, 2′-deoxyformycin, CEP-751, raltitrexed, N-propargyl-5,8-didezafolic acid, 2-chloro-2′-arabinofluoro-2′-deoxyadenosine, 2-chloro-2′-deoxyadenosine, 9-aminocamptothecin anisomycin, trichostatin, hPRL-G129R, linomide, sulfur mustard, N-methyl-N-nitrosourea, fotemustine, streptozotocin, bisplatinum, temozolomide, mitozolomide, AZQ, ormaplatin, CI-973, DWA2114R, JM216, JM335, tomudex, azacitidine, cytrabincine, gemcitabine, 6-mercaptopurine, teniposide, hypoxanthine, doxorubicine, CPT-11, daunorubicine, darubicin, epirubicine, nitrogen mustard, losoxantrone, dicarbazine, amscrine, pyrazoloacridine, all trans retinol, 14-hydroxy-retro-retinol, all-trans retinoic acid, N-(4-hydroxyphenyl) rertinamide, 13-cisretinoic acid, 3-methyl TTNEB, 9-cisretenoic acid, fludarabine, and 2-Cda.

Additional antitumor agents include adecylpenol, 20-epi-1,25-dihydroxyvitamin-D3, 5-ethynyl uracil, abiraterone, aclarubicine, acylfulvene, adozelecin, aldesleukin, ALL-TK antagonists, altretamine, ambumastine, amidox, amifostine, amino levulinic acid, anagralide, anastrozole, andrographolide, antagonist D, antarelix, anti-dorsalizing morphogenetic protein-1, antiandrogen, antiestrogen, antineoplastone, antisense oligonucleotides, aphidicolin, apoptosis gene modulators, apotosis regulators, apurinic acid, ara-cdp-dl-PTBA, arginine aminase, asulacrine, atamestine, atrimustine, axinamastine 1 and axinamastine 2, axinamastine 3, azasetron, azatoxin, azatyrosine, baccatin III derivatives, balanol, BCR/ABL antagonist, benzochlorins, benzoylsaurosporine, beta lactam derivatives, beta-alethine, perillyl alcohol, phenozenomycin, phenyl acetate, phosphatase inhibitors, picibanil, pilocarbine and salts or analogs thereof, pirarubucin, piritrexim, placetin A, placetin B, plasminogen activator inhibitor, platinum complex, phenyl ethyl isothiocyanate and analogs thereof, platinum compounds, platinum triamine complex, podophylotoxin, porfimer sodium, porphyromycin, propyl bis acridones, mTOR inhibitors, prostaglandins J2, protease inhibitors, protein A based immune modulators, PKB inhibitors PKC inhibitors, microalgal, protein tyrosine phosphatase inhibitors, purine nucleoside phosphorylase inhibitors, purpurins, pyrazoloacridines, pyridoxylated haemoglobin polyoxyethylene conjugate, raf antagonists, raltitrexed, ramosetron, ras farnesyl protein tranaferase inhibitors, ras inhibitors, ras-GAP inhibitors, ratellitptine demethylated, Rhenium Re186 etidronate, rhizoxine, ribozyme, RII retinide, rogletimide, rosagliatazone and analogs and derivatives thereof, rohitukine, romurtide, roquinimex, rubiginone B1, ruboxyl, safingol, saintopin, SarCNU, sarcophytol A, sargrmostim, sdi 1 mimetics, semustine, senescence derived inhibitor 1, sense oligonucleotide, signal transduction inhibitors, signal transduction modulators, single chain antigen binfing protein, sizofiran, sobuzoxane, sodium borocaptate, sodium phenyl acetate, solverol, somatomedin binding protein, sonermin, sparfosic acid, spicamycin D, spiromustin, splenopentine, spongistatin 1, squalamine, stem cell inhibitor, stem cell division inhibitor, stipiamide, stromelysin, sulfinosine, superactive vasoactive intestinal peptide antagonists, suradista, siramin, swainsonine, synthetic glycosaminoglycans, tallimustine, tamoxifen methiodide, tauromustine, tazarotene, tacogalan sodium, tegafur, tellurapyrilium, telomerase inhibitors, temoporfin, tmeozolomide, teniposide, tetrachlorodecaoxide, tetrazomine, thaliblastine, thalidomide, thiocoraline, thrombopoetin and mimetics thereof, thymalfasin, thymopoetin receptor agonist, thymotrinan, thyroid stimulating harmone, tin ethyl etiopurpin, tirapazamine, titanocene and salts thereof, topotecan, topsentin, toremifene, totipotent stem cell factors, translation inhibitors, tretinoin, triacetyluridine, tricribine, trimetrexate, triptorelin, tropisetron, turosteride, tyrosine kinase inhibitors, tyrphostins, UBC inhibitors, ubenimex, urogenital sinus derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozol, zanoterone, zeniplatin, zilascorb and zinostatin.

Additional antitumor agents include antiproliferative agents (e.g., piritrexim isothiocyanate), antiprostatic hypertrophy agents (sitogluside), Benign prostatic hyperplasia therapy agents (e.g., tomsulosine, RBX2258), prostate growth inhibitory agents (pentomone) and radioactive agents: fibrinogen 1125, fludeoxyglucose F18, flurodopa F18, insulin 1125, iobenguane 1123, iodipamide sodium 1131, iodoantipyrine 1131, iodocholesterol 1131, iodopyracet 1125, iofetamine HCL 1123, iomethin 1131, iomethin 1131, iothalamate sodium 1125, iothalamate 1131, iotyrosine 1131, liothyronine 1125, merosproprol Hg197, methyl ioodobenzo guanine (MMG-I131 or MIBGI123) selenomethionine Se75, technetium Tc99m furifosmin, technetium Tc99m gluceptate, Tc99m biscisate, Tc99m disofenin, TC99m gluceptate, Tc99m lidofenin, Tc99m mebrofenin, Tc99m medronate and sodium salts thereof, Tc99m mertiatide, Tc99m oxidronate, Tc99m pentetate and salts thereof, Tc99m sestambi, Tc99m siboroxime, Tc99m succimer, Tc99m sulfur colloid, Tc 99m teboroxime, Tc 99m tetrofosmin, Tc99m tiatide, thyroxine 1125, thyroxine 1131, tolpovidone 1131, triolein 1125, treoline 1125, and treoline 131.

Another category of antitumor agents is anticancer supplementary potentiating agents, e.g., antidepressant drugs (imipramine, desipramine, amitryptyline, clomipramine, trimipramine, doxepin, nortryptyline, protryptyline, amoxapine, and maprotiline), or non-trycyclic anti-depressant drugs (sertaline, trazodone and citalopram), Ca²⁺ antagonists (verapamil, nifedipine, nitrendipine and caroverine), calmodulin inhibitors (prenylamine, trifluroperazine and clomipramine), amphotericin B, triparanol analogs (e.g., tomoxifene), antiarrythmic drugs (e.g., quinidine), antihypertensive drugs (e.g., resepine), thiol depleters (e.g., buthionine and sulofoximine) and multiple drug resistance reducing agents such as cremaphor EL.

In certain embodiments, antitumor agents include annoceous acetogenins, ascimicin, rolliniastatin, guanocone, squamocin, bullatacin, squamotacin, axanes, baccatin, and taxanes (Paclitaxel and docetaxel). In certain embodiments, antitumor agents include immune checkpoint inhibitors, such as but not limited to: monoclonal antibodies that target PD-1 and/or PD-L1, such as, but not limited to, pembrolizumab (KEYTRUDA®), lambrolizumab (MK-3475), nivolumab (BMS-936558/MDX-1106/ONO-4538, OPDIVO®), pidilizumab, CT-011, AMP-224, AMP-514, BMS-936559/MDX-1105, MPDL3280A, MSB0010718C and MEDI-4736; monoclonal antibodies that target CTLA-4, such as but not limited to ipilimumab (YERVOY®).

In certain embodiments, antitumor agents include anti-CD20 mAB, rituximab, rituxan, tositumoman, Bexxar, anti-HER2, trastuzumab, Herceptin, MDX20, antiCA125 mAB, antiHE4 mAB, oregovomab mAB, B43.13 mAB, Ovarex, Breva-REX, AR54, GivaRex, ProstaRex mAB, MDX447, gemtuzumab ozoggamycin, Mylotarg, CMA-676, anti-CD33 mAB, anti-tissue factor protein, Sunol, IOR-C5, C5, anti-EGFR mAB, Erbitux, anti-IFR1R mAB, MDX-447, anti-17-1A mAB, edrecolomab mAB, Panorex, anti-CD20 mAB (Y-90 lebelled), ibritumomab tiuxetan (IDEC-Y2B8), zevalin, and anti-idiotypic mAB.

In certain embodiments, antitumor agents include a serine/threonine kinase inhibitor, a phosphatidyl inositol 3-kinase-like (PIKK) protein kinase inhibitor, a DNA dependent protein kinase (DNA-PK) inhibitor (e.g. NU7441, NU7026, KU-0060648, PI-103, PIK75, PP121 and DMNB), an Ataxia Telangiectasia Mutated (ATM) inhibitor (e.g. KU-55933, KU-60019 and CP-466722), an Ataxia Telangiectasia and Rad3 Related (ATR) inhibitos (BEZ235, VE-821 and AZD6738) or an ATM/ATR inhibitor (e.g. Wartmannin, CGK 733, Torin 2 and VE-822).

In certain embodiments, antitumor agents include Poly (ADP-ribose) polymerase (PARP) inhibitors, such as but not limited to olaparib, Iniparib, Talazoparib, Niraparib, Veliparib, Rucaparib, and 3-aminobenzamide.

As used herein, the term “ATM” refers to ataxia telangiectasia mutated.

As used herein, the term “BRCA1” refers to breast cancer 1, early onset.

As used herein, the term “BRCA2” refers to breast cancer 2, early onset.

As used herein, the term “PALB2” or “FANCN” refers to partner and localizer of BRCA2.

As used herein, the term “PTEN” refers to phosphatase and tensin homolog and is a tumor suppressor.

As used herein, the term “RAD50” and “RAD51” are exemplary of DNA repair protein.

As used herein, the term “cancer” is defined as disease characterized by the rapid and uncontrolled growth of aberrant cells. Cancer cells can spread locally or through the bloodstream and lymphatic system to other parts of the body. Examples of various cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, endometrial cancer, glioma, glioblastoma multiforme, neuroblastoma, melanoma, cholangiocarcinoma and the like. The term “cancer” as used herein, should be construed to include any malignant tumor including, but not limited to, carcinoma (any cancer of epithelial origin) or sarcoma (any mesenchymal neoplasm that arises in bone and soft tissues).

As used herein the terms “cholangiocarcinoma” refers to a form of bile duct cancer composed of mutated epithelial cells. Mutations in IDH1 and IDH2 are among the most common genetic alterations in cholangiocarcinoma, particularly in intrahepatic cholangiocarcinoma (IHCC).

In one aspect, the terms “co-administered” and “co-administration” as relating to a subject refer to administering to the subject a compound of the invention or salt thereof along with a compound that may also treat the disorders or diseases contemplated within the invention. In one embodiment, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, oral, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, pleural, peritoneal, subcutaneous, epidural, otic, intraocular, and/or topical administration.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “DNA repair protein-deficient cell” refers to a cell wherein one or more of the proteins involved in the pathway(s) for DNA repair is absent, expressed at a low level, less active (by virtue of mutation(s), truncation(s), deletion(s), partial inactivation, inhibition by small molecules and/or other proteins, and so forth) or inactive, as compared to a control cell. In certain embodiments, the one or more proteins that is/are absent, expressed at a low level, less active or inactive belongs to the DNA double-strand break (DSB) repair pathway.

As used herein, the terms “effective amount,” “pharmaceutically effective amount” and “therapeutically effective amount” refer to a sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

The term “anti-tumor effective amount” as used herein refers to an amount of a compound that treats or prevents cancer.

“Glioma” as used herein is a type of brain tumor. Gliomas can be classified as grade Ito grade IV on the basis of histopathological and clinical criteria established by the World Health Organization (WHO). WHO grade I gliomas are often considered benign.

Gliomas of grade II or III are invasive, progress to higher-grade lesions. Grade IV tumors (glioblastomas) are the most invasive form. Exemplary brain tumors include, e.g., astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma); oligodendroglial tumor (e.g., oligodendroglioma, and anaplastic oligodendroglioma); oligoastrocytic tumor (e.g., oligoastrocytoma, and anaplastic oligoastrocytoma); ependymoma (e.g., myxopapillary ependymoma, and anaplastic ependymoma); medulloblastoma; primitive neuroectodermal tumor, schwannoma, meningioma, meatypical meningioma, anaplastic meningioma; and pituitary adenoma (Balss et al., Acta Neuropathol 116:597-602 (2008); Yan et al., N Engl J Med. 360 (8):765-73 (2009)).

As used herein, the term “Isocitrate dehydrogenase (IDH)” refers to a class of enzymes that catalyze the oxidative decarboxylation of isocitrate to 2-oxoglutarate (i.e., α-ketoglutarate, α-KG). These enzymes belong to two distinct subclasses, one of which utilizes NAD(+) as the electron acceptor and the other NADP(+). Five isocitrate dehydrogenases are known in the art: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer.

IDH1 (isocitrate dehydrogenase 1 (NADP+), cytosolic) is also known as IDH; IDP; IDCD; IDPC or PICD. The protein encoded by this gene is the NADP(+)-dependent isocitrate dehydrogenase found in the cytoplasm and peroxisomes and plays a role in NADPH production. The nucleotide and amino acid sequences of IDH1 are well known in the art and has been reported in several species. In some embodiments, the human nucleotide and amino acid sequences of IDH1 are GenBank entries NM-005896.2 and NP-005887.2 respectively. The human IDH1 gene encodes a protein of 414 amino acids (Nekrutenko et al., Mol. Biol. Evol. 15:1674-1684 (1998); Geisbrecht et al., J. Biol. Chem. 274:30527-30533 (1999); Sjoeblom et al., Science 314:268-274 (2006)).

IDH2 (isocitrate dehydrogenase 2 (NADP+), mitochondrial) is also known as IDH; IDP; IDHM; IDPM; ICD-M; or mNADP-IDH. The protein encoded by this gene is the NADP(+)-dependent isocitrate dehydrogenase found in the mitochondria. It plays a role in intermediary metabolism and energy production. In some embodiments, the human nucleotide and amino acid sequences of IDH2 can be found as GenBank entries NM-002168.2 and NP-002159.2 respectively. The human IDH2 gene encodes a protein of 452 amino acids (Huh et al., Submitted (NOV-1992) to the EMBL/GenBank/DDBJ databases; and The MGC Project Team, Genome Res. 14:2121-2127 (2004)).

A non-mutant (e.g., wild type) IDH1 catalyzes the oxidative decarboxylation of isocitrate to α-KG thereby reducing NAD (NADP+) to NADP (NADPH), e.g., in the forward reaction:

Isocitrate+NAD+(NADP+)→α-KG+CO2+NADH(NADPH)+H+

A mutant IDH has the ability to convert α-KG to 2-HG. In some embodiments, a mutant IDH1 can arise from a mutation of His, Ser, Cys or Lys, or any other at residue 132 as described previously by Yan et al. (Yan et al., N Engl J Med 360:765-773 (2009)). A mutant IDH2 can arise from a mutation of Gly, Met or Lys, or any other at residue 172 (Yan et al., N Engl J Med 360:765-773 (2009)). Exemplary mutations include the following: R132H, R132C, R132S, R132G, R132L, and R132V.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound and/or composition cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, “metastasis” refers to the distant spread of a malignant tumor from its sight of origin. Cancer cells may metastasize through the bloodstream, through the lymphatic system, across body cavities, or any combination thereof.

As used herein, “proliferating” and “proliferation” refer to cells undergoing mitosis.

The terms “patient” and “subject” and “individual” are used interchangeably herein, and refer to any animal, or cells thereof, whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions.

The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof. Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable pharmaceutically acceptable base addition salts of compounds of the present invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

The term “prevent,” “preventing” or “prevention,” as used herein, means avoiding or delaying the onset of symptoms associated with a disease or condition in a subject that has not developed such symptoms at the time the administering of an agent or compound commences.

By the term “specifically bind” or “specifically binds,” as used herein, is meant that a first molecule preferentially binds to a second molecule (e.g., a particular receptor or enzyme), but does not necessarily bind only to that second molecule.

A “therapeutic” treatment is a treatment administered to a subject who exhibits signs of pathology, for the purpose of diminishing or eliminating those signs.

As used herein, the term “treat,”“treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.1, 5.3, 5.5, and 6. This applies regardless of the breadth of the range.

### Description

The invention includes compositions and methods for treating or preventing cancer in a subject. In one aspect the invention provides a method of treating a subject suffering from a cancer wherein the cells thereof contain an IDH1 or IDH2 mutation. The method comprises administering to the subject at least one compound comprising a DNA repair inhibitor. In another aspect, when the cells of the cancer do not contain an IDH mutation, the method of the invention comprises administering to the subject a therapeutically effective amount of at least one compound selected from the group consisting of 2-hydroxyglutarate (2-HG), a derivative of 2-HG, any variant and any mixtures thereof In a further aspect, the invention provides a pharmaceutical composition comprising an anti-tumor effective amount of at least one compound comprising a 2-hydroxyglutarate (2-HG), a derivative of 2-HG, any variant and any mixtures thereof In some embodiments, the derivative of 2-HG is (2R)-octyl-α-hydroxyglutarate (e.g. AGI-5198, Cayman Chemicals; octyl-(R)-2HG) or a mixture of octyl-(R)-2HG with ester derivatives.

### Methods

The invention includes a method of treating or preventing cancer in a subject in need thereof, wherein the cells in the cancer comprise an IDH1 or IDH2 mutation.

As described elsewhere herein, mutant IDH converts α-KG to 2-KG. In some embodiments, the activity of the mutated IDH leads to an increased level of 2-HG in a subject. Accumulation of 2-HG in the subject may be harmful to the subject. In some embodiments, elevated levels of 2-HG lead to and/or is predictive of the presence of cancer in a subject. Such cancers include a cancer of the central nervous system (e.g., brain tumor, glioma or glioblastoma multiforme (GBM)) or a leukemia (e.g. acute myelogenous leukemia).

Notwithstanding the aforementioned methods, the invention also includes a method of treating a cancer in a subject, where the cells of the cancer do not contain an IDH mutation. The method comprises administering to the subject a therapeutically effective amount of at least one compound selected from the group consisting of 2-HG, a derivative of 2-HG, any variant and any mixtures thereof In some embodiments, the derivative of 2-HG is (2R)-octyl-α-hydroxyglutarate. In certain embodiments, the methods of the invention further comprises administering to the subject a therapeutically effective amount of at least one compound comprising a DNA repair inhibitor, whereby the cancer is treated or prevented in the subject.

In certain embodiments, the at least one compound inhibits DNA double strand break repair in the cancer. In certain embodiments, the at least one compound inhibits a serine/threonine kinase, a PIKK protein kinase, a DNA-PK, an ATM or an ATR.

In other embodiments, the at least one compound inhibits homology recombination DNA repair in the cancer. In yet other embodiments, the at least one compound is a poly(ADP-ribose) polymerase (PARP) inhibitor selected from the group consisting of olaparib, Iniparib, Niraparib, Veliparib, Rucaparib, 3-aminobenzamide and BMN-673 (Talazoparib).

In further embodiments, the at least one compound is an alpha-ketoglutarate-dependent dioxygenase A or B (KDM4A or KDM4B) inhibitor selected from the group consisting of DMOG, NSC 636819, PK 118 310, NCGC 00247751, NCGC 00244536, NCGC 00247743, IXO1, Disulfiram and JIB04. PARP inhibitors and KDM4A/KDM4B inhibitors are well known and commonly used in the art. Any of these inhibitors or any derivatives therefrom are suitable for use in the methods of the invention.

In certain embodiments, the subject is further administered at least one additional antitumor agent. In other embodiments, the antitumor agent is selected from the group consisting of topoisomerase inhibitors; alkylating agents; nitrosoureas; antimetabolites; antitumor antibiotics; antimicrotubule agents; hormonal agents; DNA strand break inducing agents; epidermal growth factor (EGF) receptor inhibitors and ant-EGF receptor antibodies; AKT inhibitors; mTOR inhibitors; CDK inhibitors; receptor tyrosine kinase (RTK) inhibitors; ribonucleotide reductase inhibitors; serine/threonine kinase inhibitors, phosphatidyl inositol 3-kinase-like (PIKK) protein kinase inhibitors, DNA dependent protein kinase (DNA-PK) inhibitor, Ataxia Telangiectasia Mutated (ATM) inhibitors, Ataxia Telangiectasia and Rad3 Related (ATR) inhibitors and immune checkpoint inhibitors. In yet other embodiments, administration of the at least one compound and at least one additional antitumor agent is synergistic. In yet other embodiments, the at least one compound and at least one additional antitumor agent are coadministered to the subject. In yet other embodiments, the at least one compound and at least one additional antitumor agent are coformulated so that they are combined into a single pharmaceutical compound.

In certain embodiments, the subject is further administered radiation therapy. In other embodiments, administration of the at least one compound and the radiation therapy is synergistic.

In certain embodiments, the at least one compound is administered to the subject through a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, pleural, peritoneal, subcutaneous, epidural, otic, intraocular, and topical.

In certain embodiments, the cancer comprises breast cancer, prostate cancer, ovarian cancer, cervical cancer, skin cancer, pancreatic cancer, colorectal cancer, renal cancer, liver cancer, brain cancer, glioma, glioblastoma multiforme, melanoma, lymphoma, acute myeloid leukemia (AML), cholangiocarcinoma, leukemia, lung cancer, endometrial cancer, head and neck cancer, sarcoma, multiple myeloma and/or neuroblastoma. In other embodiments, the cancer comprises glioma or leukemia. In yet other embodiments, the cancer comprises cells defective in at least one protein selected from the group consisting of BRCA1, BRCA2, PTEN, ATM, ATR, PALB2, FANCD2, RAD50, RAD51, other components of the homology dependent DNA repair pathway or the non-homologous end joining pathway or other components that mediate or regulate DNA repair.

In other aspects, when cancer cells carry an IDH mutation, the cancer comprises brain, head and neck cancer, glioma, meningioma, glioblastoma multiforme, lymphoma, leukemia, AML, cholangiocarcinoma, multiple myeloma and neuroblastoma. In yet other aspects, the cancer comprises glioma or AML.

In certain embodiments, the subject is a mammal. In other embodiments, the mammal is a human.

### Compounds and Compositions

In certain embodiments, the compounds of the invention, or a salt or solvate thereof, comprise at least one compound selected from the group consisting of 2-HG, a derivative of 2-HG, any variant and any mixtures thereof In certain embodiments, the derivative of 2-HG is (2R)-octyl-α-hydroxyglutarate (e.g. AGI-5198, Cayman Chemicals; octyl-(R)-2HG) or a mixture of octyl-(R)-2HG with ester derivatives.

Pharmaceutical compositions comprising at least one compound of the invention, as well as at least one pharmaceutically acceptable carrier, are also contemplated in the invention. Pharmaceutical compositions comprising at least one compound of the invention and at least one additional antitumor agent, as well as at least one pharmaceutically acceptable carrier, are also contemplated in the invention.

### Combination Therapies

In certain embodiments, the compounds of the invention are useful in the methods of the invention in combination with at least one additional antitumor compound. This additional compound may comprise compounds identified herein or compounds, e.g., commercially available compounds, known to treat, prevent or reduce the symptoms of cancer.

In one aspect, the present invention contemplates that a compound useful within the invention may be used in combination with a therapeutic agent such as an antitumor agent, including but not limited to a chemotherapeutic agent, an anti-cell proliferation agent or any combination thereof.

For example, any conventional chemotherapeutic agents of the following non-limiting exemplary classes are included in the invention: topoisomerase inhibitors; alkylating agents; nitrosoureas; antimetabolites; antitumor antibiotics; antimicrotubule agents; hormonal agents; DNA strand break inducing agents; EGF receptor inhibitors and anti-EGF receptor antibodies; AKT inhibitors; mTOR inhibitors; CDK inhibitors; receptor tyrosine kinase (RTK) inhibitors; ribonucleotide reductase inhibitors; serine/threonine kinase inhibitors, phosphatidyl inositol 3-kinase-like (PIKK) protein kinase inhibitors, DNA dependent protein kinase (DNA-PK) inhibitors, Ataxia Telangiectasia Mutated (ATM) inhibitors, and Ataxia Telangiectasia and Rad3 Related (ATR) inhibitors.

Topoisomerase inhibitors include etoposide, camptothecin, topotecan, irrinotecan, teniposide, and mitoxantrone.

Alkylating agents are so named because of their ability to add alkyl groups to many electronegative groups under conditions present in cells, thereby interfering with DNA replication to prevent cancer cells from reproducing. Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Antimetabolites prevent incorporation of bases into DNA during the synthesis (S) phase of the cell cycle, prohibiting normal development and division. Non limiting examples of antimetabolites include drugs such as 5-fluorouracil, 6 mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include dactinomycin, daunorubicin, doxorubicin, idarubicin, mitomycin-C, and mitoxantrone.

Antimicrotubule agents include plant alkaloids that inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids. The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

DNA strand break inducing agents include bleomycin, doxarubicine, daunorubicine, idarubicine, and mitomycin.

Miscellaneous agents include chemotherapeutics such as hydroxyurea, L-asparaginase, and procarbazine that are also useful in the invention.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof In other specific aspects, the Bcl-2 family member is, for example, Bax, Bak, Bcl-Xs, Bad, Bid, Bik, Hrk, Bok, or a combination thereof.

In certain embodiments, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof In other embodiments, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or a combination thereof.

A “synergistic effect” as used herein relates to an effect of two or more compounds on a subject where the effect of the combination is greater than the sum of their individual effects. A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively. In some aspects, the effect of treatment of the subject in need thereof with a combination of the compound of the invention an additional antitumor agent (e.g. PARP inhibitor) is.

### Kits

The invention includes a kit comprising at least a compound of the invention, an applicator, and an instructional material for use thereof. The instructional material included in the kit comprises instructions for preventing or treating a cancer with cells containing an IDH1IDH2 mutation contemplated within the invention in a subject. The instructional material recites the amount of, and frequency with which, the at least one compound of the invention should be administered to the subject. In other embodiments, the kit further comprises at least one additional antitumor agent.

### Patient Selection and Monitoring

Described herein are compositions and methods for treating or preventing a cancer in a subject in need thereof wherein the cancer contains an IDH1 or IDH2 mutation. In some embodiments, the compositions and methods of this inventions are useful for a subject who is at risk of developing cancer associated with a mutation in an IDH enzyme (e.g., IDH1 and/or IDH2)). In some embodiments, a subject is selected for treatment with a compound described herein based on a determination that the subject has a mutant IDH enzyme. In some embodiments, the subject or a sample (e.g., tissue or bodily fluid) therefrom is evaluated for the presence or amount of a substrate, cofactor and/or product of the IDH enzyme. The presence and/or amount of substrate, cofactor and/or product can correspond to the wild-type/non-mutant activity or can correspond to the mutated form of the enzyme. Exemplary bodily fluids that can be used to identify and evaluate the IDH enzyme include but are not limited to amniotic fluid surrounding a fetus, aqueous humour, blood (e.g., blood plasma), serum, Cerebrospinal fluid, cerumen, chyme, sperm, female ejaculate, interstitial fluid, lymph, breast milk, mucus (e.g., nasal drainage or phlegm), pleural fluid, pus, saliva, sebum, semen, serum, sweat, tears, urine, vaginal secretion, or vomit.

In some embodiments, a subject can be evaluated for carrying an IDH mutation (IDH1 and/or IDH2) using any methods known to one skilled in the art by way of non-limiting example: magnetic resonance, chemical assays (e.g., High performance liquid chromatography (HPLC)), sequencing and PCR. In other embodiments, the subject can be evaluated for the presence of and/or an elevated amount of 2-hydroxyglutarate (2-HG) relative to the amount of 2-HG present in a subject who does not have a mutation in IDH.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The therapeutically effective amount or dose of a compound of the present invention depends on the age, sex and weight of the patient, the current medical condition of the patient and the progression of a disease or disorder contemplated in the invention.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In one embodiment, patients require intermittent treatment on a long-term basis upon any recurrence of symptoms.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal and the like. In many cases, it is preferable to include isotonic agents, for example, sugars, sodium chloride, or polyalcohols such as mannitol and sorbitol, in the composition.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for any suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., analgesic agents.

Suitable compositions and dosage forms include, for example, dispersions, suspensions, solutions, syrups, granules, beads, powders, pellets, liquid sprays for nasal or oral administration, dry powder or aerosolized formulations for inhalation, and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds of the invention, and a further layer providing for the immediate release of a medication for treatment of a disease or disorder. Using a wax/pH-sensitive polymer mix, a gastric insoluble composition may be obtained in which the active ingredient is entrapped, ensuring its delayed release.

### Parenteral Administration

For parenteral administration, the compounds may be formulated for injection or infusion, for example, intravenous, intramuscular or subcutaneous injection or infusion, or for administration in a bolus dose and/or continuous infusion. Solutions, suspensions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 2003/0147952, 2003/0104062, 2003/0104053, 2003/0044466, 2003/0039688, and 2002/0051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 min up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 min, about 20 min, or about 10 min and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 min, about 20 min, or about 10 min, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Materials and Methods

### Cell Lines and Culture Conditions

Human immortalized astrocytes were cultured in high glucose Dulbecco's Modified Eagle Medium (DMEM) containing 10% tetracycline-free fetal bovine serum (FBS;

Clontech Laboratories and Thermo Fisher Scientific). HCT116 cells (Horizon) were cultured in McCoy's 5A Medium containing FBS. U2OS DR-GFP cells have been described previously (Bindra et al., Nucleic acids research 41, e115 (2013)). DLD1 wild-type and BRCA2-null cells (Horizon) were cultured in Roswell Park Memorial Institute Medium (RPMI; Gibco) and FBS. Glioma lines LN18 and LN229 (ATCC) were cultured in DMEM/Nutrient Mixture F-12 (DMEM/F12) and FBS (Thermo Fisher Scientific). Purchased cell lines were appropriately authenticated by vendors prior to use. All cell lines were certified free of mycoplasma by our institution's pathogen testing facility. All cells were maintained at 37° C. with 5% CO₂.

### Chemicals Used

(2R)-octyl-a-hydroxyglutarate (Cayman), (2S)-octyl-α-hydroxyglutarate (Cayman), AGI-5198 (Selleck), IDHC227 (Xcessbio), AG-120(Selleck), octyl-a-ketoglutarate (Cayman), BMN-673 (Selleckchem), cisplatin (CDDP; Tocris), olaparib (Selleckchem), TH287 (Sigma), TCS2312 (Tocris), BEZ-235 (Selleckchem), KU5593 (Selleckchem), AZD7762 (Tocris), MK-1775 (Selleckchem), NU-7441 (Selleckchem), 20 VE822 (Selleckchem), Methyl methanesulfonate (MMS; Sigma), temozolomide (TMZ; Sigma), lomustine (CCNU; Selleckchem), irinotecan (Selleckchem), mitomycin C (MMC; Cayman), razoxane (Santa Cruz Biotech), aphidicolin (Sigma), recombinant human FLT-3, SCF, TPO, IL-3, and IL-6 (Gemini).

### Generation of Lentiviral Particles and Transduction of HEL Cells

pSLIK-IDH1 (Addgene plasmid # 66802), pSLIK-IDH1-R132H (Addgene plasmid # 66803), pSLIK-IDH2 (Addgene plasmid # 66806), and pSLIK-IDH2-R172K (Addgene plasmid # 66807) (Lewis et al., 2014, Molecular cell 55, 253-263). To generate lentiviral particles for transduction, 293T cells were plated in a T175 flask to 70% confluence and incubated overnight. The next day they were transfected with 23 μg psPAX2, 14 pCMV-VSVG, and 32 μg pSLIK transfer vectors using Lipofectamine 2000 (Life Technologies). Viral supernatant was collected 24 and 36 h after transfection, filtered with a 45 μM filter, and aliquoted for storage at −80° C. HEL cells were transduced by spinfection at 1000×g for 90 min at 30° C. in media containing polybrene at 4 μg/mL.

### CRISPR/Cas9 Gene Editing

IDH1-R132H/+mutant astrocytes were generated using CRISPR/Cas9 gene editing (Ran et al., Nature protocols 8, 2281-2308 (2013)). Guide RNAs were designed to the IDH1 locus using the MIT-Broad design tool (crispr.mit.edu/), and single-stranded donor DNA containing the requisite mutation to convert Arg-132 to His plus a silent Bcl1 restriction endonuclease site was synthesized. These were cotransfected along with a separate plasmid containing the Cas9 cDNA using the Amaxa Nucleofector system (Lonza). Targeted cleavage was confirmed by T7 endonuclease assay. Following limiting dilution, single-cell colonies were screened for the heterozygous mutation by high-resolution melt analysis utilizing the silent Bcl1 restriction endonuclease site. TOPO clones were generated using the TOPO-TA Cloning Kit per manufacturer's protocol (Cat. #450071; Thermo Fisher Scientific). Sanger sequencing of both endogenous DNA as well as TOPO clones confirmed the presence of the heterozygous mutation.

### Western Blotting

WT and IDH1 R132H mutant astrocyte cells were lysed in AZ lysis buffer (50 mM Tris, 250 mM NaC1, 1% Igepal, 0.1% SDS, 5 mM EDTA, 10 mM Na₂P₂O₇, 10 mM NaF) supplemented with Protease Inhibitor Cocktail (Roche) on ice for 20 min. Cellular debris was cleared by centrifugation and lysate protein concentration was quantified using the DC Protein Assay (Bio-Rad). Lysate containing 80 μg protein was subjected to SDS-PAGE in a Mini-PROTEAN TGX 4-20% gradient gel (Bio-Rad) and then transferred to nitrocellulose or PVDF membrane. The following primary antibodies were used for western blot analysis: rabbit monoclonal anti-IDH1 (D2H1, Cell Signaling), rabbit polyclonal anti-IDH1-R132H (H09, Dianova), mouse monoclonal anti-BRCA1 (D9, Santa Cruz Biotechnology), mouse monoclonal anti-FANCD2 (103, Abcam), mouse monoclonal anti-RAD51 (14B4, Novus Biologicals), rabbit polyclonal anti-SMC1 (Bethyl), and mouse monoclonal anti-Vinculin (SPM227, Abcam). Band intensities were quantified using ImageJ software and normalized to Vinculin expression.

### HGDH-Mediated Enzyme Assay

Protocol as previously published by Balss et al. (Acta Neuropathol 124, 883-891 (2012)). Briefly, 25 μL of conditioned media from cells in culture was added to 75 μL of reaction mix containing 100 mM HEPES pH 8.0, Diaphorase 1.0 U/mL, resazurin 5μM, NAD+100 μM, HGDH 1.0 μg/mL in 96-well plates. Reactions were performed in three technical replicates. The reactions were well-mixed and incubated at room temperature in the dark for 30 minutes. Following incubation, they were read on a plate reader in fluorescence mode (BioTek), with excitation of 510 nm and emission of 590 nm. Values reported are mean ±standard deviation.

### NAD+ Assay

NAD+ levels were quantified using the NAD/NADH Quantification Kit per manufacturer's protocol (Cat. #MAK037; Sigma). Reactions were performed in three technical replicates, and values reported are mean ±standard deviation.

### Clonogenic Survival Assay

Cells in culture were irradiated at varying doses of ionizing radiation. Four to six hours after irradiation, they were trypsinized, washed, counted, and seeded in 6-well plates in triplicate at 3-fold dilutions ranging from 9000 to 37 cells per well. Depending on colony size, these plates were kept in the incubator for 10 to 14 days. Following incubation, colonies were washed in PBS, stained with Crystal Violet, and counted and quantified. Each experiment reported was performed twice, and values reported from representative experiments are mean±standard deviation.

### cl Comet Assay

Neutral comet assays were performed per manufacturer's protocol (Trevigen). Briefly, cells were trypsinized, washed with PBS and suspended in LM Agarose (Trevigen). Neutral electrophosesis was conducted at 21V for 1 hour in the CometAssay Electrophoresis System (Trevigen). Data were collected with an EVOS FL microscope (Advanced Microscopy Group) and analyzed using AutoComet software (TriTek Corporation). For each experiment, three biological replicates each with >50 cells were measured, and reported values are mean±SEM.

### Luciferase Based Reporter Assays for HDR and NHEJ

The HR luciferase reporter was generated by cloning an inactivating I-SceI recognition site into the BstBI site 56 amino acids into the firefly luciferase gene in the gWIZ.Luciferase vector (Gelantis), and cloning a promoterless copy of the firefly luciferase open reading frame 700 base pairs downstream in reverse orientation as a donor template for HR. A DSB in the firefly luciferase gene was induced by I-SceI digestion and confirmed by electrophoresis. Linearized plasmid was transfected into cells to measure HR as a function of luciferase activity (firefly luciferase activity can only be restored by HR, which removes the inactivating I-SceI site). To assay NHEJ, a HindIII-mediated DSB was generated between the promoter and the coding region of the firefly luciferase gene in the pGL3-Control Vector (Promega) and confirmed by electrophoresis. Linearized plasmid is transfected, and repair of this DSB by NHEJ restores firefly luciferase activity. All reporter assays were performed in 12-well format by seeding 7×10⁴ cells per well 24 hours before transfection and transfecting 1 μg of reporter or positive control vector and 50 ng Renilla luciferase vector per well. For HR cells were analyzed 48 hours after reporter transfection and for NHEJ cells were analyzed 24 hours after reporter transfection. Luciferase activity was measured using the Dual Luciferase Reporter Assay System (Promega) for all samples and normalized to Renilla luciferase signal to control for transfection efficiency and to a positive control luciferase expression vector gWIZ.luciferase for HR and pG13_control for NHEJ. Statistical significance was determined by biologic triplicate. Values reported are mean±SEM.

### Cell Cycle Analysis

Cells were fixed in triplicate with ice cold 70% ethanol for 2 h, pelleted and washed with PBS, then suspended in 500 μl of PI/RNAse Staining buffer (BD Biosciences) or 1:10000 DAPI in PBS and analyzed by flow cytometry. Data was analyzed using FlowJo software and presented as a representative plot.

### U2OS DR-GFP Reporter Assays

These reporter assays are carried out as previously described (Stachelek et al., Molecular cancer research: MCR 13, 1389-1397 (2015)). Briefly, after 10 days of culture in (2R)-Octyl-alpha-hydroxyglutarate (Cayman), 1×10⁶ cells were transfected in triplicate with 4 μg pI-SceI using the Amaxa Nucleofector II and Nucleofection Kit V (Lonza) per manufacturer's protocol. 72 h after transfection, cells were analyzed for GFP expression using flow cytometry, and the data was analyzed using FlowJo software to calculate %GFP positive cells. Values reported are mean±SEM.

### Immunofluorescence Assays

For γH2AX and 53BP1 foci assays, 50,000 cells per well were seeded into 8-well multiwall chamber slides (Millipore) in triplicate. Cells were fixed with cold 1% Paraformaldehyde+2% Sucrose and stained overnight at 4° C. with rabbit anti-γH2A.X (1:50, Cell Signaling #2577) or rabbit anti-phospho-53BP1 (1:50, Cell Signaling #2675) in 10% BSA and 0.5% Triton X-100. Cells were then washed and stained with 1:1000 Alexa Fluor 488 goat anti-rabbit IgG (Thermo) for 1 h at room temperature, and then stained with 1:10000 DAPI. Slides were imaged on the EVOS FL microscope (Advanced Microscopy Group) and analyzed with CellProfiler (http://cellprofiler.org/). For Cyclin A staining, immunofluorescence was performed as previously described (Surovtseva et al., 2016, Journal of the American Chemical Society 138, 3844-3855). Foci data is presented as the mean of 3 biological replicates+/−SEM with >200 cells analyzed per replicate.

### Gene Expression Microarray Analysis

As described in Bai et al. (Nature genetics 48, 59-66 (2016)). High-quality RNAs (RNA integrity number (RIN) ≥7.5) from 9 embryonic brain samples and 30 IDH1-wildtype and 84 IDH1-mutant glioma samples were used for gene expression array analysis, on the HumanHT-12 v4 Expression BeadChip (Illumina, BD-103-0204). Gene expression data were processed using limma and sva packages in R, including background correction, quantile normalization, and batch effect removal (Shi et al., Nucleic acids research 38, e204 (2010); Leek et al., Bioinformatics (Oxford, England) 28, 882-883 (2012)). For genes of interest, their mRNA signal intensities in IDH1-mutant gliomas were compared to that of IDH1-wildtype samples. P values were calculated by two-sided Wilcoxon rank-sum test.

### Analysis of The Cancer Genome Atlas (TCGA) Lower Grade Glioma Data

IDH1 and IDH2 mutation data and BRCA1, FANCD2, RADS51, XRCC4, XRCC5/KU80, and LIG4 mRNA expression Z-scores (RNA Seq V2 RSEM) for all 283 complete tumor samples in the TCGA Provisional Brain Lower Grade Glioma dataset generated by the TCGA Research Network (cancergenome.nih.gov) were downloaded via cBioPortal (Cerami et al., Cancer discovery 2, 401-404 (2012); Gao et al., Science signaling 6, pll (2013)). DNA repair gene expression in IDH1 and IDH2 mutant samples was compared to that in IDH1 and IDH2 wild-type samples. Box plot whiskers indicate 5^{th} and 95^{th} percentiles. Statistical analyses by unpaired student t-test were preformed using GraphPad Prism Version 6.0a for MAC OS X (GraphPad Software).

### Reverse Transcription-Quantitative PCR (RT-qPCR)

Total RNA from wild-type and IDH1 R132H mutant astrocyte cells was prepared using the RNeasy Mini Kit (Qiagen). The optional on-column DNase digestion was performed with the RNase-Free DNase Set (Qiagen) to eliminate genomic DNA. Complementary DNA (cDNA) was synthesized using 750 ng RNA in the High Capacity cDNA Reverse Transcription Kit (Applied Biosystems). The resulting cDNA was diluted 1:5 and used in triplicate PCRs containing TaqMan Gene Expression Assay premixed primers and probes for BRCA1, FANCD2, RAD51, and ACTB and TaqMan Universal PCR Master Mix (Applied Biosystems). An Mx300P RT-PCR system (Strategene) was used to measure fluorescence intensity in real-time and to calculate cycle thresholds. Cₜ values were normalized to ACTB and relative expression was calculated using the −ΔΔCₜ method.

### Growth Delay Assay

Cells were plated in 96-well black-walled plates (Costar) at a concentration of 2.5 k/well and allowed to adhere at room temperature for 60 minutes prior to return to the incubator. For growth delay assays containing octyl-(R)-2HG, cells were cultured with indicated concentration for 10 days prior to plating. 24 hours later, the media was changed and indicated drugs dissolved in either DMSO or DMF (cisplatin only) were added in quadruplicate at varying concentrations. For synergy experiments, cells were replica-plated and drugs added in single wells at the indicated concentrations. At 96 hours after the addition of drugs, cells were washed in PBS, fixed in 70% ethanol, and stained with Hochst at 1 μg/mL. The plates were then imaged on a Cytation 3 automated imager (BioTek), and cells were counted using CellProfiler (cellprofiler.org/). Each experiment was performed three times and values from a representative experiment are reported as mean±SEM. For synergy experiments, experiments were analyzed for synergistic interactions using Combenefit (www.cruk.cam.ac.uk/research-groups/jodrell-group/combenefit).

### Long-Term Colony Forming Assay

Cells were counted and diluted in media containing various concentration of drug. They were then immediately seeded in 6-well plates in triplicate at 3-fold dilutions, ranging from 9000 to 37 cells per well. These plates were kept in the incubator for 10 to 14 days, following which they were washed in PBS, stained with Crystal Violet, and quantified. Each experiment was performed twice, and values reported are mean ±standard deviation.

### Cell Viability Assays

Adherent cells were seeded at a density of 2500 cells per well, and suspension cells at a density of 5000 cells per well in solid white 96 well plates (Costar) and incubated under indicated conditions in sextuplicate. Cell viability was assayed using the Cell Titer Glo Kit (Promega) per manufacturer's protocol and data is presented as mean+/−SEM.

### In vivo BMN 673 Efficacy Studies

All animal experiments were performed in compliance with our institution's IACUC committee. Four week-old female athymic nu/nu mice were used for all in vivo xenograft studies. Mice were quarantined for at least 1 week before experimental manipulation. Human HCT116 cells (with and without IDH1 mutation) were implanted subcutaneously (3-5×10⁶ cells in 0.1 cc PBS) in the right flank of nude mice. When tumors reached an average volume of approximately 100 mm³, mice were randomized into various treatment groups (5-7 mice/group) in each study. Animals were not randomized, but were grouped based on tumor size such that tumors were roughly equivalent in size when treatment was begun. The investigator was not blinded throughout the experiment. Mice were visually observed daily and tumors were measured three times per week by calipers to determine tumor volume using the formula:

$$V = \frac{4}{3} π \times ( \frac{length}{2} ) \times ( \frac{width}{2} ) \times ( \frac{depth}{2} ) .$$

BMN 673 was solubilized in DMSO and diluted with PBS containing 10% dimethylacetamide (Sigma-Aldrich) and 6% Solutol (Sigma-Aldrich). BMN 673 (0.33 mg/kg, 0.2 cc), or vehicle (0.2 cc) was administered by oral gavage (per os), once daily for 21 consecutive days. Mean (geometric) tumor volume (mm³) was graphed over time to monitor tumor growth. Tumor tripling time was calculated as the time required for tumors to increase in volume threefold over baseline at the time of treatment initiation. P-values were determined by Mann-Whitney U test (Stata). Kaplan-Meier curves were generated with survival fraction determined by the number of mice alive with tumor volume smaller than three times the initial pre-treatment volume. Groups were compared using the log-rank test. Values reported are geometric mean±SEM. One animal was excluded from the study due to premature death, which was not determined to be due to metastasis but rather trauma from oral gavage.

### 2HG (L- and D-Isomer) Sample Preparation

Microtubes containing cell pellets were removed from −80° C. storage and maintained on wet ice throughout the processing steps. To initiate protein precipitation, 0.5 mL of a chilled mixture of methanol, chloroform and water (7:2:1) (EMD, MA, USA) containing 2.5 μM 13C5-2HG (L27 isoform, 95% purity, NIH Common Funds Metabolomics Standards Program (MetabolomicsWorbench.org)) was added to each sample, vortexed briefly, probe sonicated for 5-10 sec, and allowed to incubate on ice for 10 min. Post-incubation, the vortex step was repeated, and samples centrifuged at 14,000 RPM, for 10 min in 4° C. Postcentrifugation, 200 μL of supernatant was transferred to an autosampler vial and taken to dryness. Samples were derivatized with Diacetyl-1-tartaric anhydride (DATAN) using the following method: 50 μL of a 50 mg/mL solution of DATAN in dichloromethane:acetic acid (4:1) was added to each dried sample, samples were capped, and incubated at 75° C. for 30 min. Vials were cooled, dried under a continuous flow of N2 for 1 hour at RT. Samples were reconstituted in 100 μL of LC-grade water prior to analysis.

### LC-MS Analysis

LC-MS analysis was performed on an Agilent system consisting of a 1290 UPLC module coupled with a 6490 QqQ mass spectrometer (Agilent Technologies, CA, USA.) Metabolites were separated on an Acquity HSS-T3, 1.8 μm, 2.1 x 50 mm column (Waters Corp, MA, USA), held at 40° C., using 2 mM ammonium formate in water, adjusted to pH 3.3 with formic acid as mobile phase A, and acetonitrile containing 0.1% formic acid as mobile phase B. The flow rate was 0.2 mL/min and 2HG D- and L-isomers were separated with an isocratic elution (99%A and 1%B) for 6 minutes. The mass spectrometer was operated in ESI-mode, monitoring transitions 363.2->147.2 and 368.3->151.2 for 2HG and 13C5-2HG respectively, with a dwell time of 1000, the collision energy 8, and cell accelerator voltage 4. A standard curve with 6 points from 0 to 20 μM of a mixture of D- and L-2HG, was created and derivatized in the same manner as the samples, along with the individual D- and L-2HG standards, for quantification and retention time confirmation purposes. Data were normalized to the internal standard prior to quantification with the standard curve, and data were further normalized to the protein levels of the cell pellets for final analysis. Data is presented as the mean of three biological replicates+/−SD.

### ¹H NMR of Tumor Extracts

Frozen excised tumor tissue (150-200 mg) was used for metabolite extraction. In short, after addition of a known amount of 2-¹³C-glycine as internal concentration standard, metabolites were extracted using hydrogen chloride/methanol, and ethanol in combination with a bead 28 mill (Omni International, GA, USA). Supernatant was lyophilized and the resulting powder re-suspended for NMR analysis in a phosphate buffer (pH 7) containing formic acid as chemical shift standard and 10% deuterium for lock signal. All NMR spectra were acquired on a 500 MHz MR spectrometer (Bruker Avance, Bruker, Billerica, Mass., USA). Proton-Observed Carbon-Edited (POCE) spectra were acquired with repetition time of 18 s and echo time of 8 ms, 16 averages and 6 repetitions at a spectral width of 10 kHz. Quantification of the 2HG concentration relied on the known amount of 2-¹³C-glycine. 1H-C4 peaks from 2HG and glutamate, and the ¹H-¹³C2 peak from glycine observed in the Proton Observed Carbon-Edited difference spectrum were fitted using a linear combination of model spectra approach with in-house written software in MATLAB (Mathworks, MA, USA).

### Statistics

Data are presented as means±SEM and compared using Student's t test, one-way ANOVA, or two-way ANOVA when appropriate. Kaplan-Meier curves were compared using the log-rank test. All tests were two-sided. Statistical analyses were carried out using GraphPad Prism software. A P value of less than 0.05 was considered statistically significant. For in vivo studies, adjusted variance was calculated within each group comparison using the “ranksum” command in Stata. Variances were within an order of magnitude of each other.

### Example 1

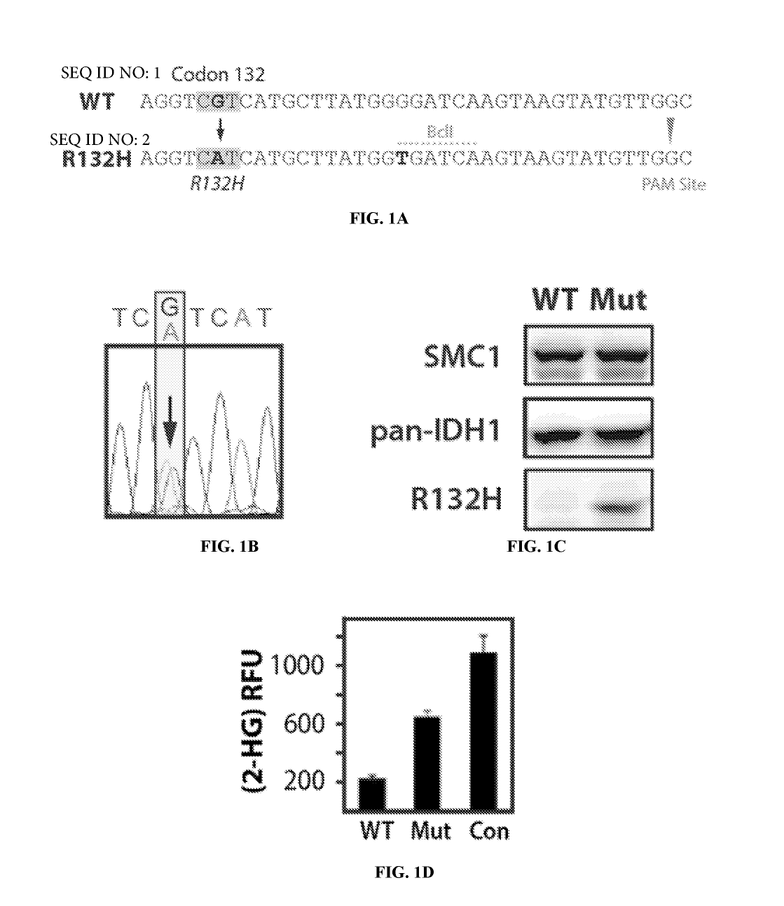

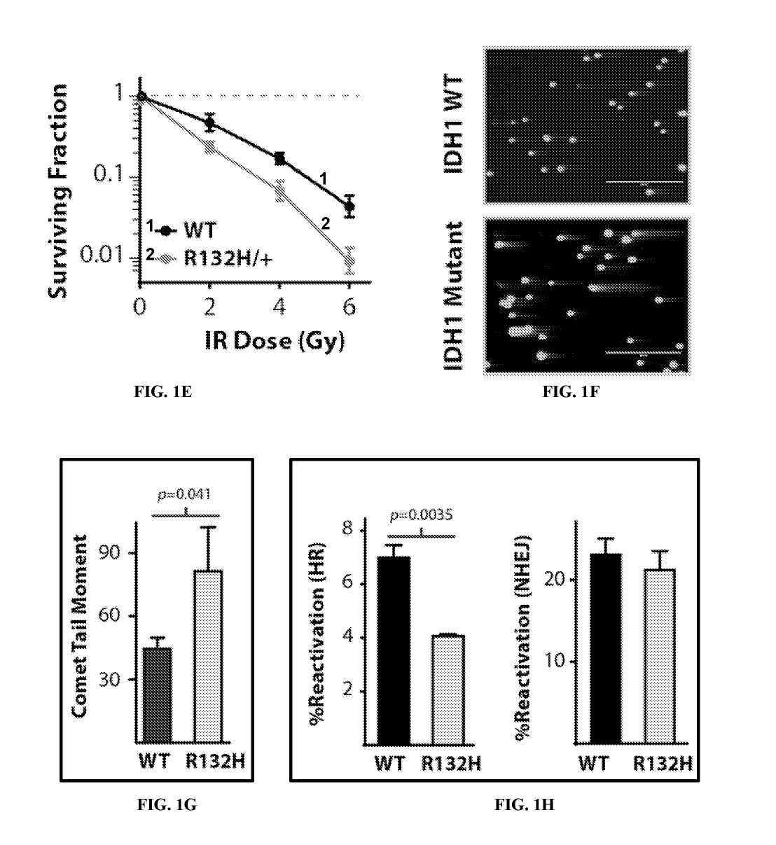

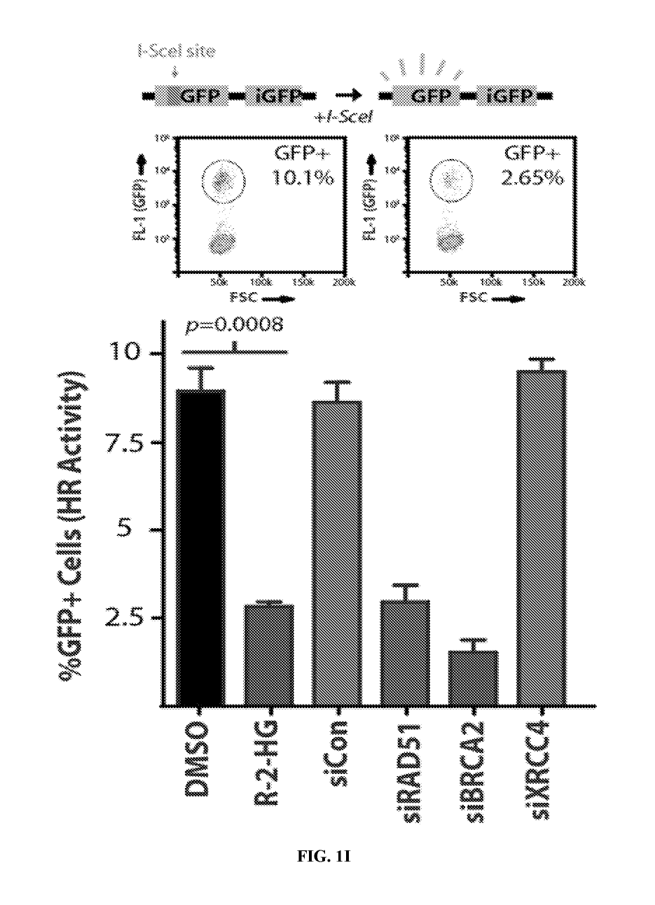

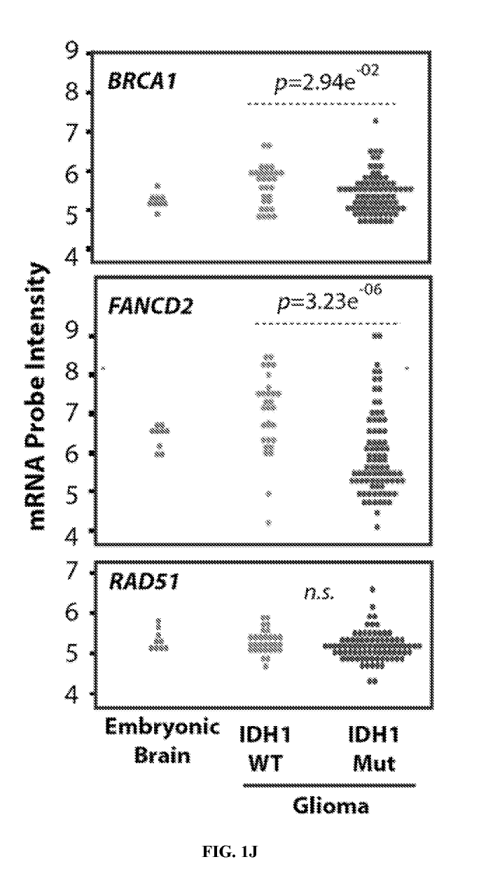

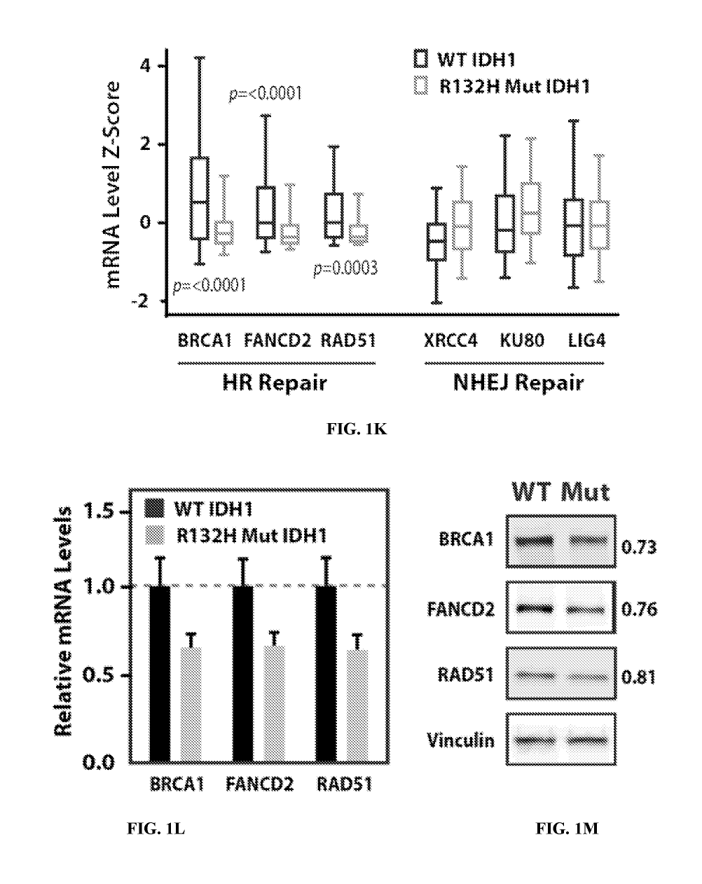

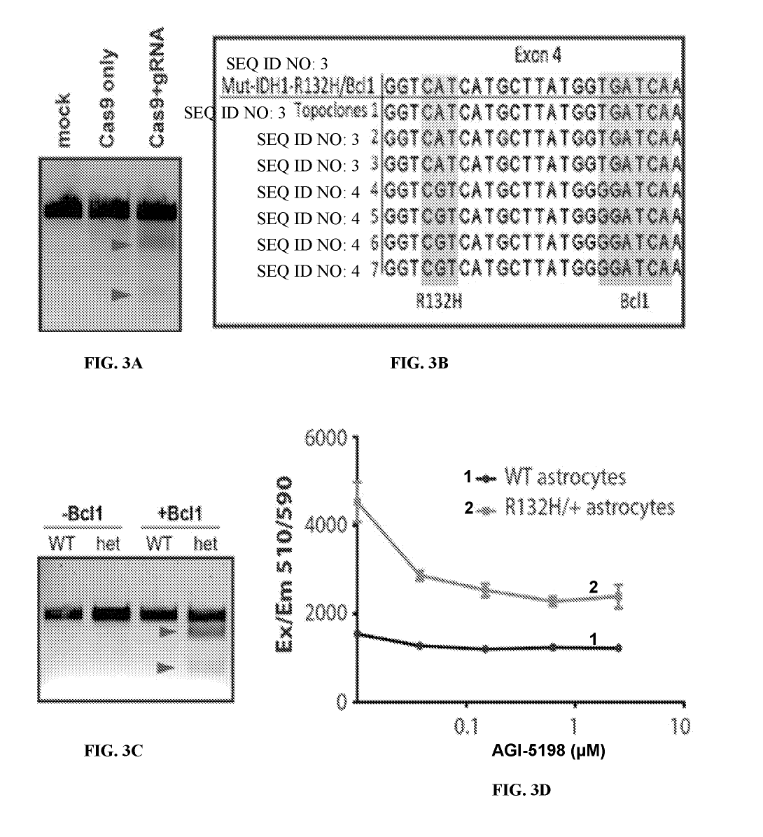

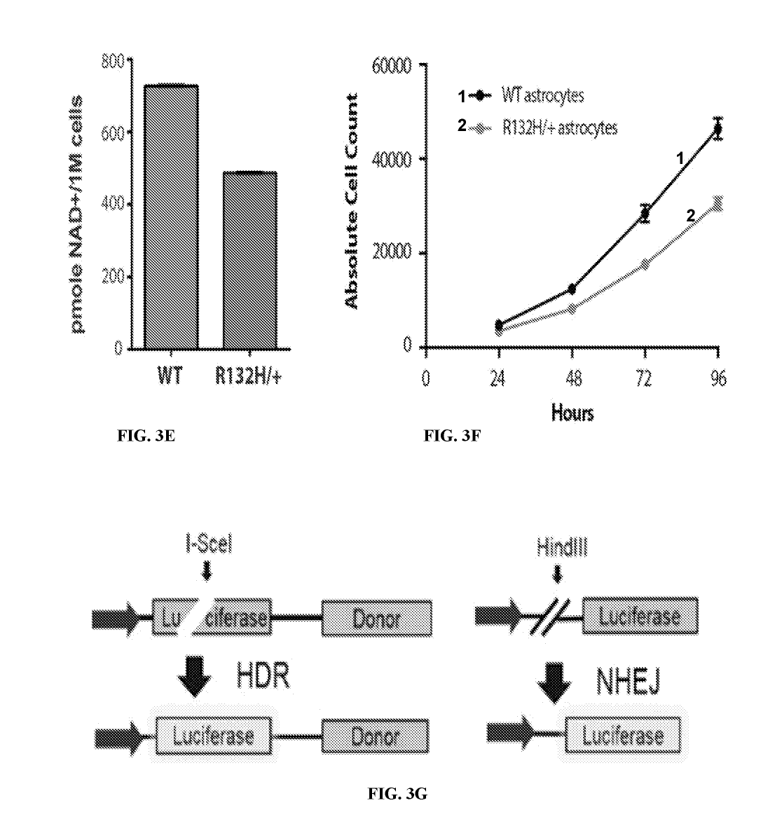

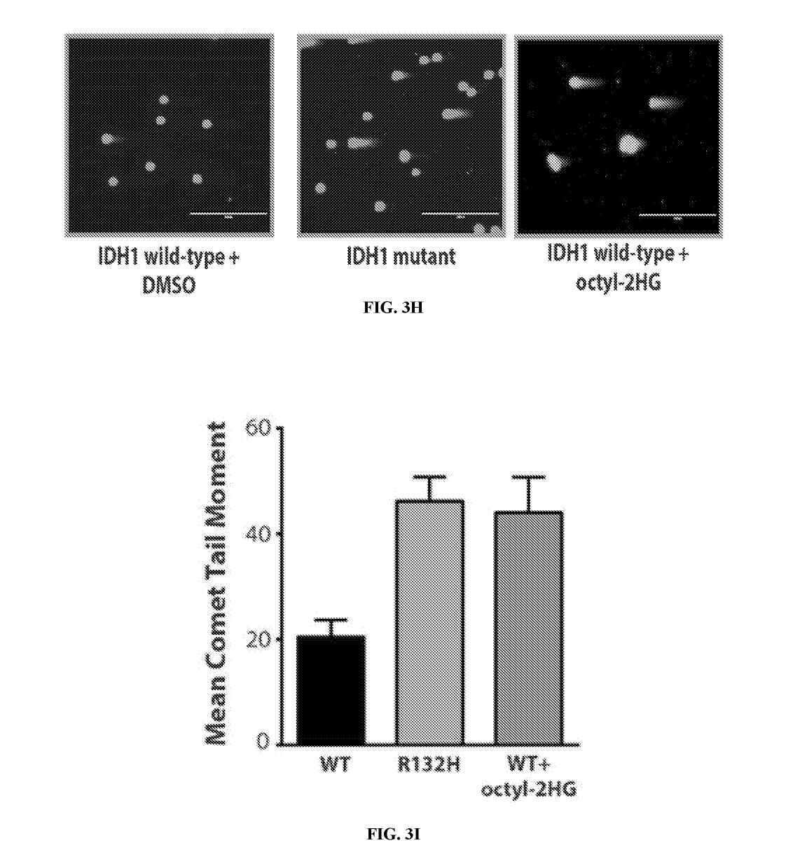

A heterozygous arginine (R) to histidine (H) mutation was first engineered at codon 132 (R132H) of the IDH1 gene at the endogenous locus in immortalized human astrocytes. A CRISPR/Cas9-based gene targeting strategy was utilized to knock-in the mutation via homologous recombination (HR; FIG. 1A). Guide RNAs (gRNAs) which directed Cas9 cleavage at the endogenous IDH1 locus (FIG. 3A) were developed together with donor DNAs to introduce the R132H mutation. A single cell clone harboring a heterozygous R132H IDH1 mutation was isolated (FIG. 1B), its heterozygosity (FIGS. 3B and 3C), and mutant IDH1 protein expression were then confirmed (FIG. 1C). This mutant cell line produced high levels of 2-HG (FIG. 1D), which was suppressed by a known IDH1 inhibitor (FIG. 3D) (Rohle et al., Science 340, 626-630 (2013)). Consistent with previous studies, the IDH1-mutant cell line of this invention had lower levels of intracellular NAD+compared to wild-type (WT) cells (FIG. 3E), (Tateishi et al., Cancer cell 28, 773-784 (2015)). Interestingly, while the present engineered mutant cell line recapitulated these major features of IDH1-mutant tumors, no significant increases in proliferation in vitro was observe. Instead, a slightly reduced growth kinetics in IDH1-mutant cells in vitro (FIG. 3F) was observed. This is the first reported human astrocyte-derived isogenic WT and mutant IDH1 mutant cell line pair, which will be useful as an in vitro model system for pre-clinical drug development efforts.

IDH1-mutant astrocytes were found to be significantly more radiosensitive than WT cells (FIG.1E). This led us to speculate that IDH1 mutations may induce an intrinsic double-strand break (DSB) repair defect in tumors. The DSB repair defect was then characterized using the comet assay to measure persistence of DSBs after IR, and also functional reporter assays to interrogate specific DNA repair pathways (FIG. 3G). IDH1-mutant astrocytes displayed a significantly reduced capacity to repair DSBs after IR exposure (FIGS. 1F-1G) and showed a marked deficiency in HR in comparison to WT cells, while no differences were observed in the other major DSB repair pathway, non-homologous end-joining (NHEJ; FIG. 1H). In addition, constitutively high levels of DSBs were observed in IDH1-mutant cells even in the absence of IR, which further suggested a baseline defect in DSB repair (FIGS. 3H-3I). Mechanistically, treatment with 2-HG alone was found be able to recapitulate the IDH1-associated HR defect (FIG. 11, FIGS. 3H-3I). Suppression of HR by 2-HG was dramatic, and it approached levels seen with siRNAs targeting two key HR genes, RAD51 and BRCA2.

IDH1 mutations are known to induce profound gene expression changes via epigenetic remodeling (Losman et al., Genes Dev 27, 836-852 (2013)). DSB repair gene expression patterns were assessed herein in the context of IDH1 mutations as a possible mechanism to explain the observed HR defect. Indeed, gene expression analysis of IDH1-WT and -mutant low-grade gliomas revealed substantial decreases in several HR-associated genes, including BRCA1, FANCD2, and RAD51 (FIG. 1J) (Bai et al., Nature genetics 48, 59-66 (2016)). This finding was also independently validated in a cohort of patients with IDH1-WT and -mutant tumors using data from The Cancer Genome Atlas (TCGA; FIG. 1K). Importantly, there was minimal gene expression difference in NHEJ-related factors in this analysis, suggesting that IDH1 mutations induce a selective suppression of the HR axis. Additionally, these same genes were confirmed to be repressed at the mRNA level in IDH1-mutant astrocytes by quantitative PCR (FIG. 1L), and these differences were detectable at the protein level (FIG. 1M).

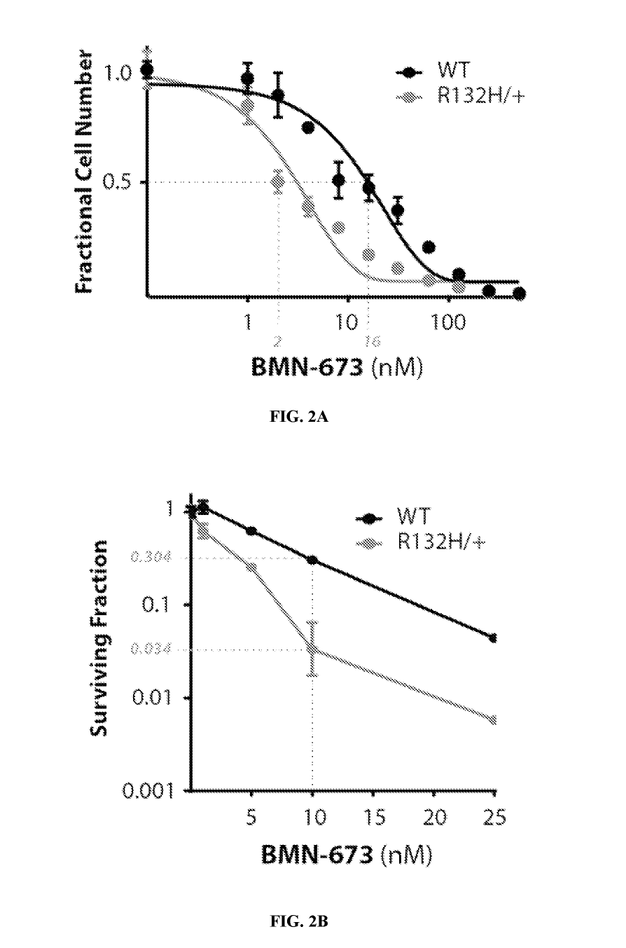

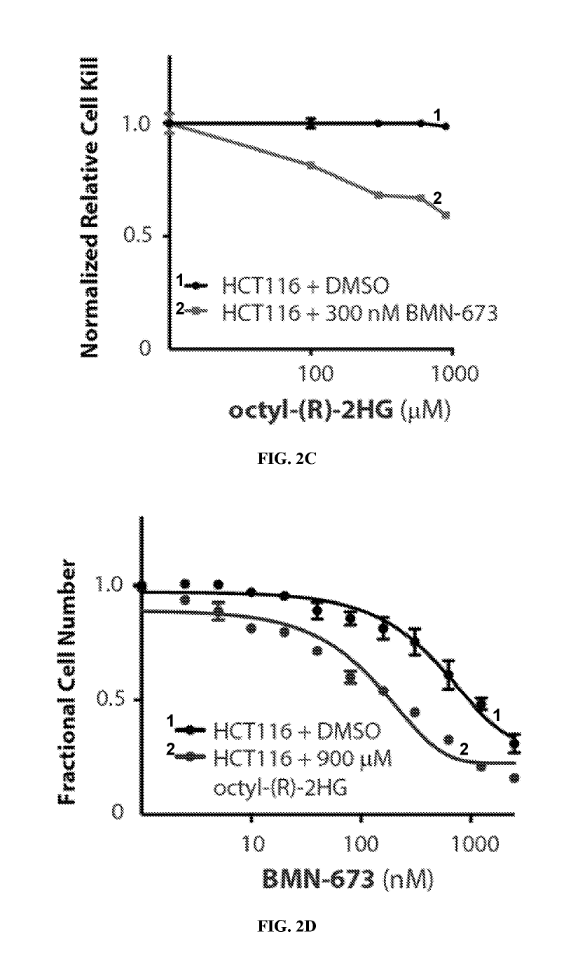

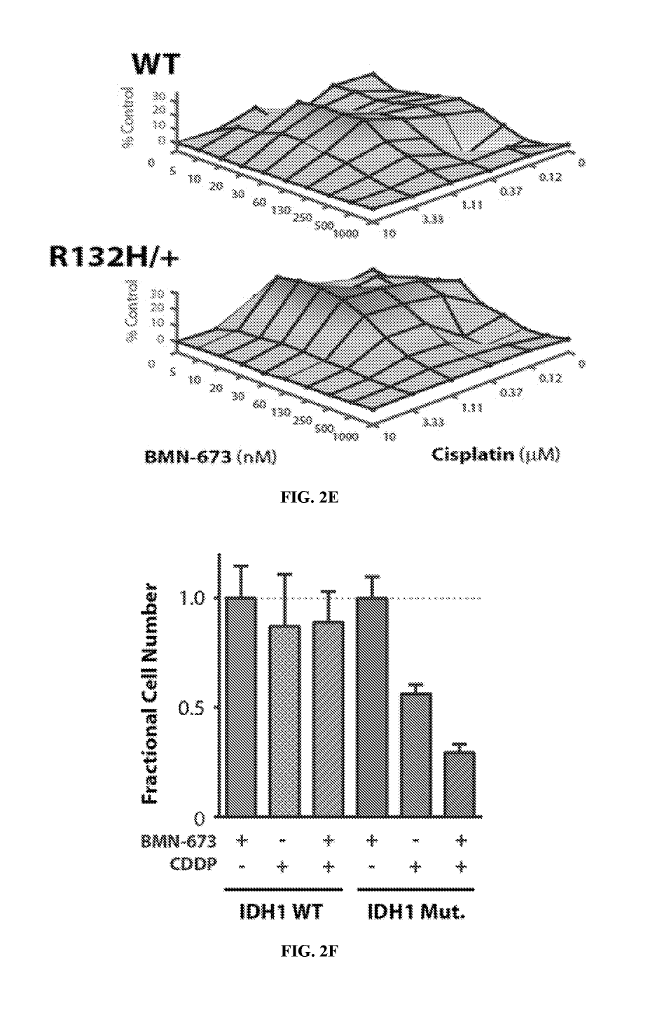

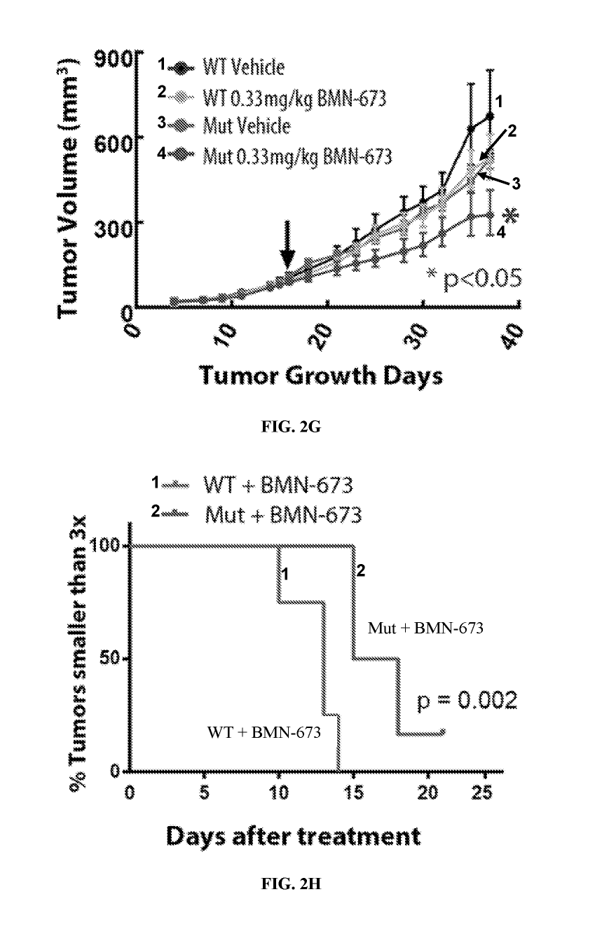

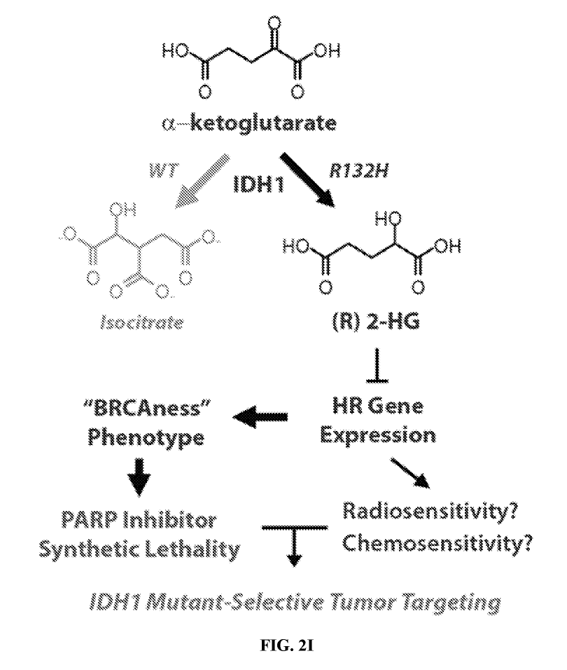

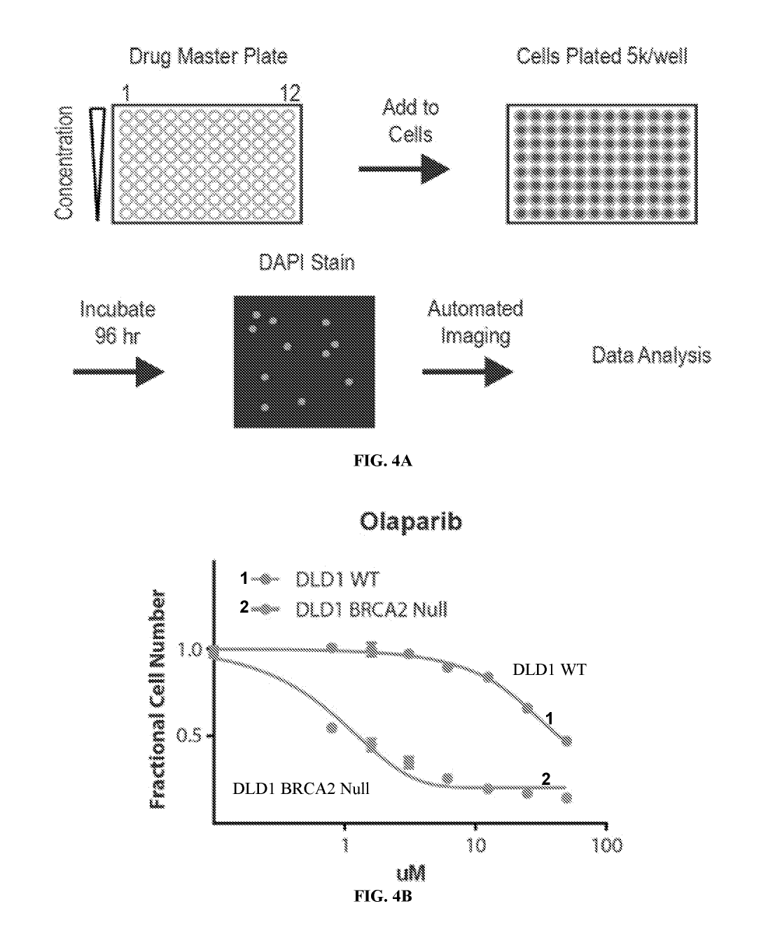

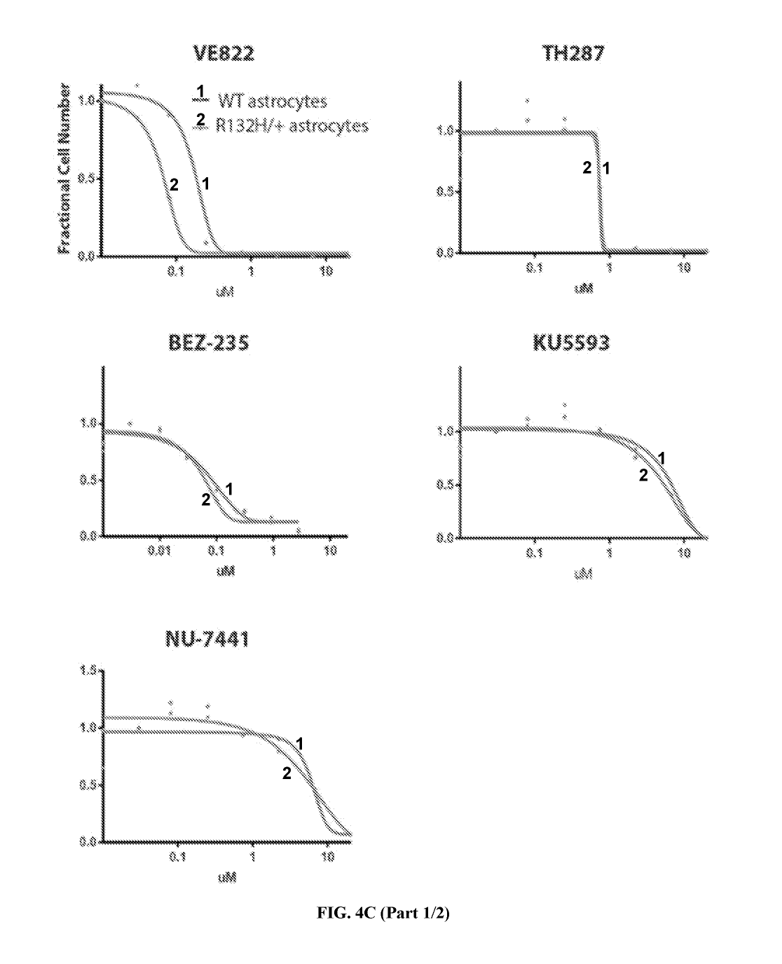

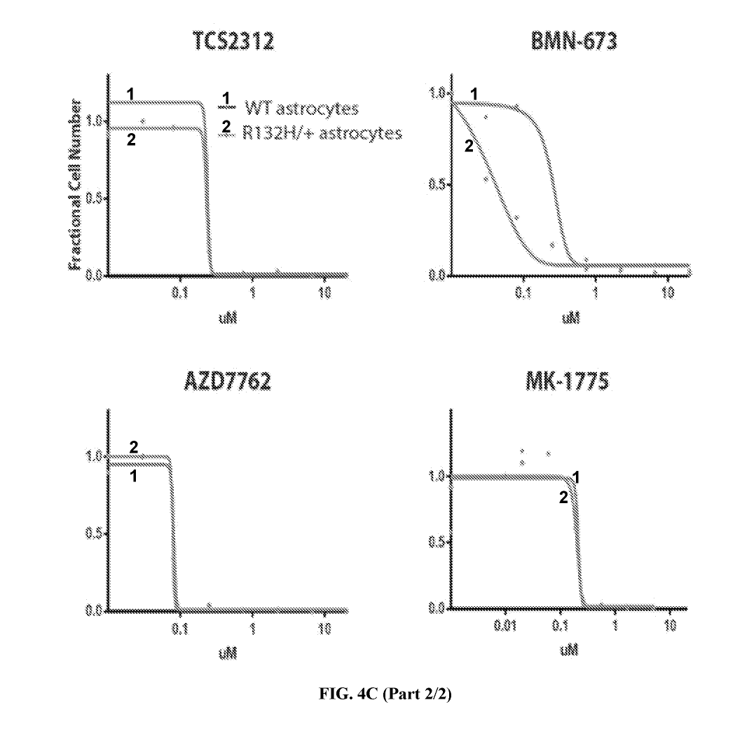

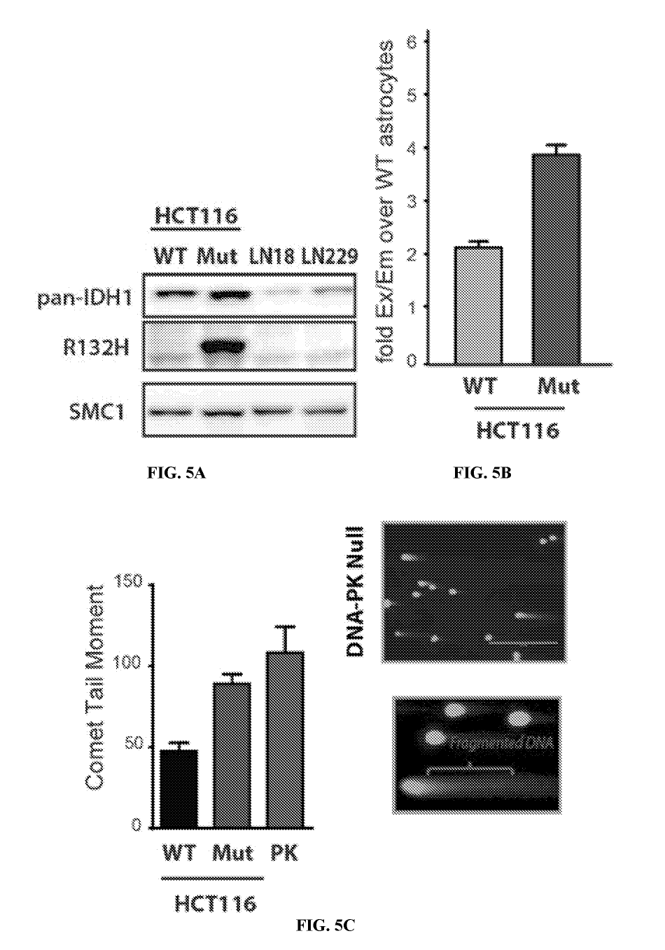

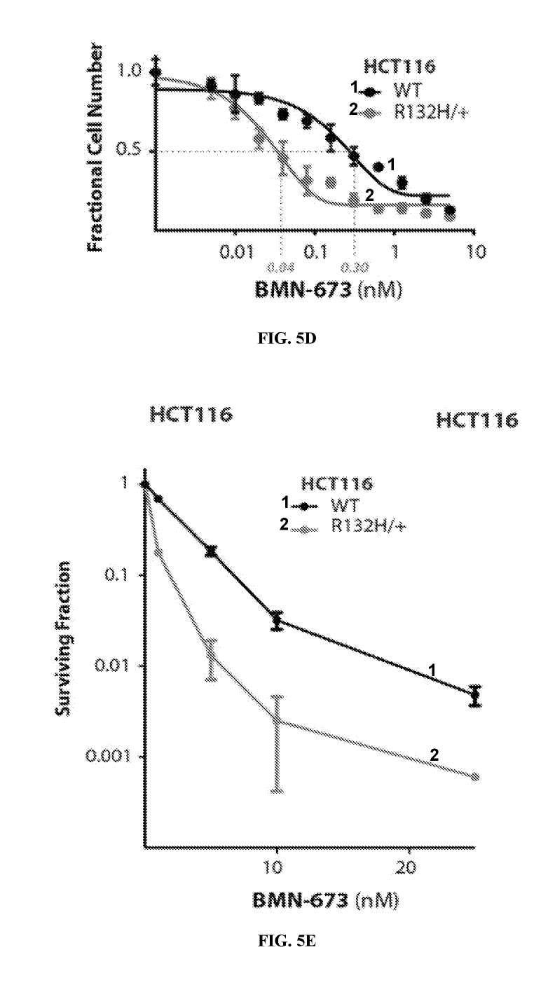

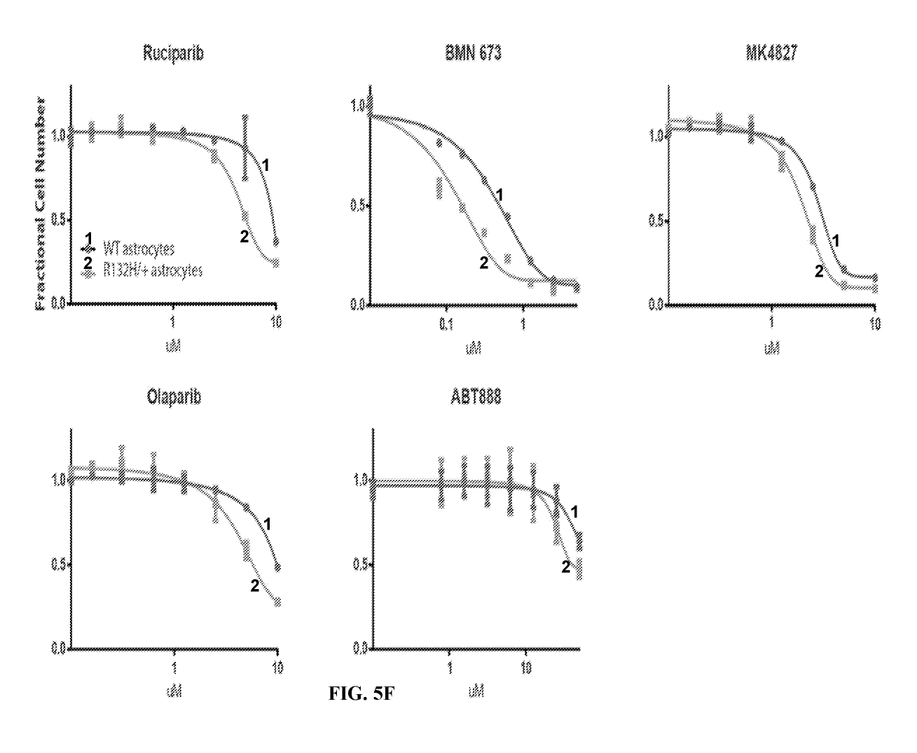

It is now well established that HR defects confer sensitivity to DNA repair pathway inhibition via synthetic lethal interactions. Thus, IDH1-WT and -mutant cells of this invention were profiled against a focused collection of small molecule DNA damage response and DSB repair inhibitors, using a high-throughput, short-term cell growth inhibition assay (FIGS. 4A-4C). These studies revealed a synthetic lethal interaction between Poly (ADP-Ribose) Polymerase (PARP) inhibitors and IDH1-mutant astrocytes (FIG. 2A). IDH1 mutation status also conferred exquisite sensitivity to the PARP inhibitor, BMN-673, in clonogenic survival assays, with a 10-fold decrease in cell survival compared to WT cells at a dose of 10 nM (FIG. 2B). A similar HR defective phenotype was observed with an enhanced PARP inhibitor sensitivity in other matched isogenic IDH1-WT and mutant cell lines, including an isogenic HCT116 cell line pair (FIGS. 5A-5E). IDH1-mutant cells were also confirmed to be sensitive to several unique PARP inhibitors, which confirmed the specificity of the interaction (FIG. 5F). Once again, treatment of WT cells with 2-HG was shown to be able to recapitulate the enhanced PARP inhibitor sensitivity seen in IDH1-mutant cells (FIGS. 2C-2D).

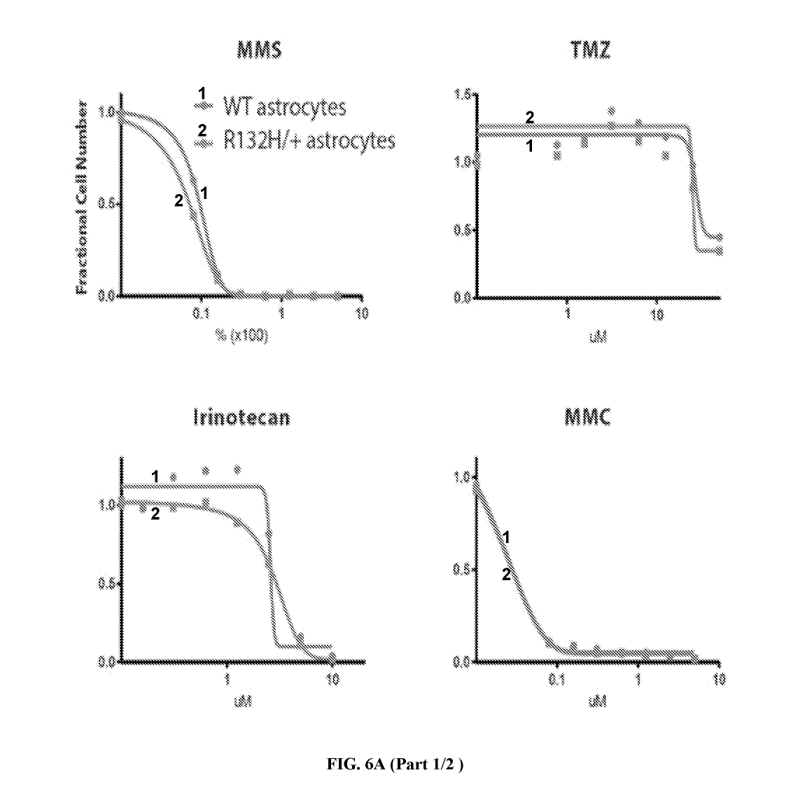

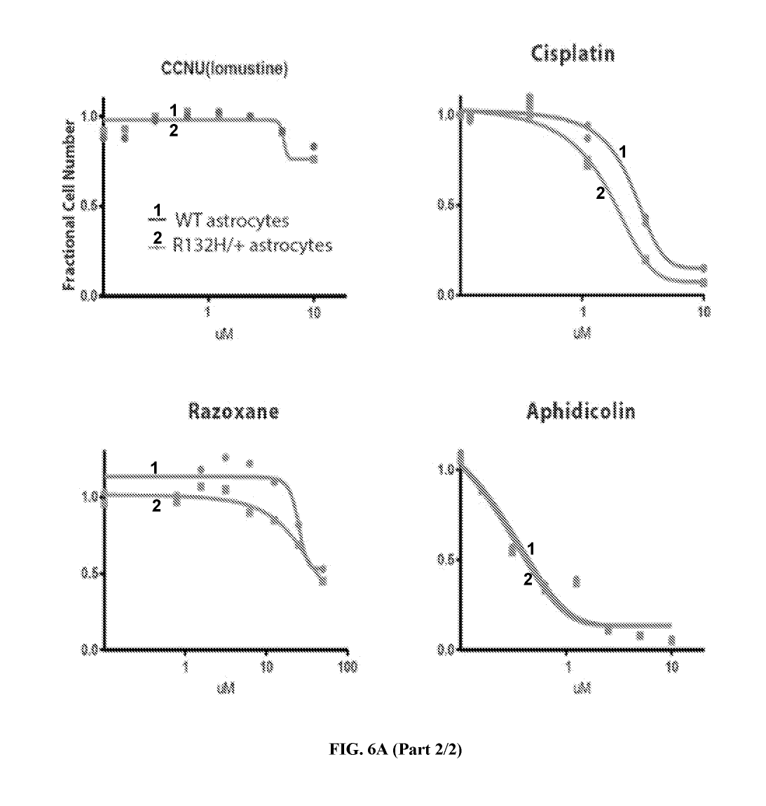

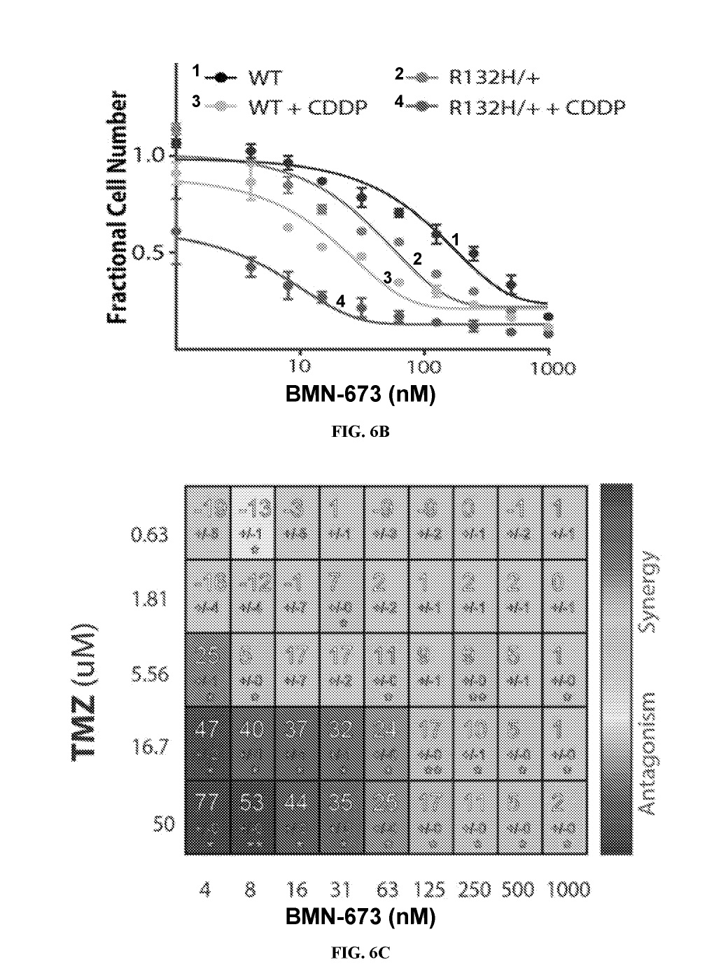

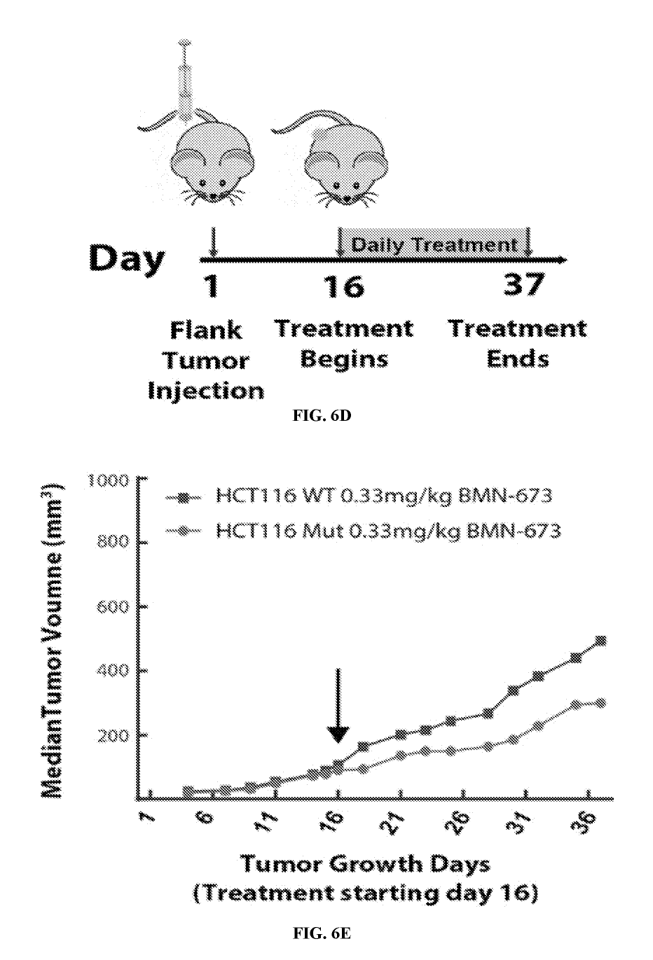

Synergistic interactions have been reported when PARP inhibitors are combined with DNA damaging agents in both HR-deficient and -proficient tumors, including platinum and temozolomide (TMZ) (Loser et al., Molecular cancer therapeutics 9, 1775-1787 (2010); Michels et al., Oncogene 33, 3894-3907 (2014)). A combination of these agents was tested in the isogenic cell line pairs of the present invention. First, IDH1-WT and -mutant cells were profiled in short-term growth inhibition assays with a collection of DNA damaging agents to assess baseline differential sensitivity. These studies revealed modest, but detectable sensitization with cisplatin, MMS, and irinotecan as single agents (FIG. 6A). However, a dramatic synergistic interaction was observed between cisplatin and BMN-673 in IDH1-mutant tumor cells (FIG. 2E-F and FIG. 6B). Similar results but of a lesser magnitude were observed with TMZ (FIG. 6C).

Then the extent to which IDH1 mutant-dependent PARP inhibitor sensitivity could be recapitulated in vivo was tested using a subcutaneous xenograft tumor model. To this end, HCT116 IDH1-WT and -mutant tumor cells were implanted into the flanks of nude mice, and treated them with BMN-673 versus vehicle alone. A significant growth delay was observed in IDH1-mutant tumors upon treatment of mice with BMN-673, with minimal effects in IDH1-WT tumors (FIGS. 2G-2H, and FIGS. 6D-6E).

### Example 2

### The 2HG-Induced BRCAness Occurs Via Inhibition of Specific αKG-Dependent Dioxygenases

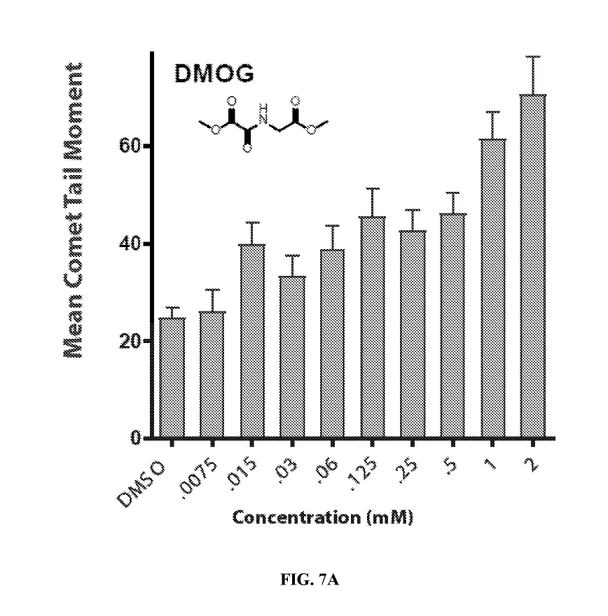

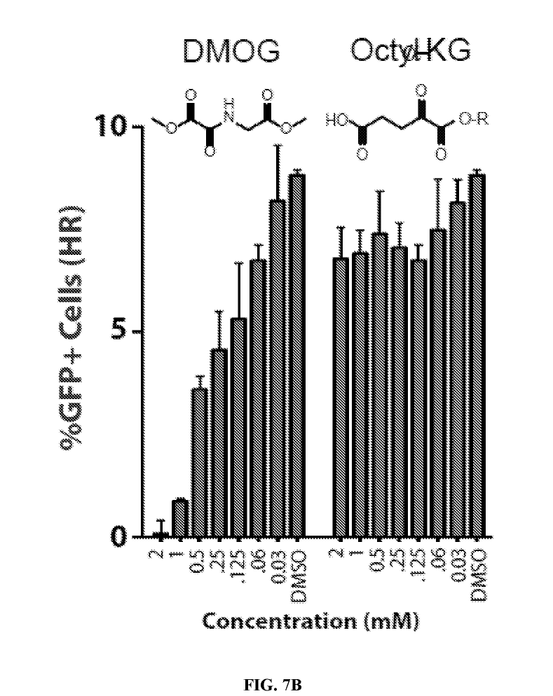

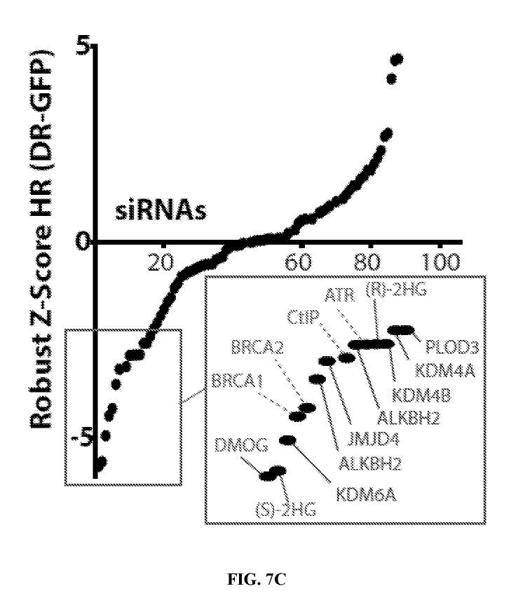

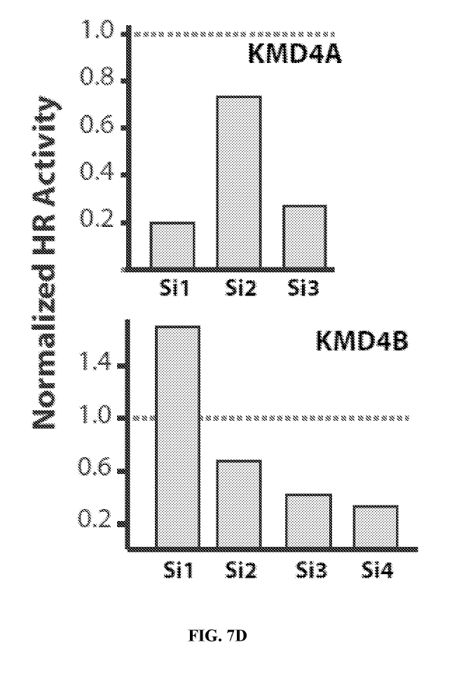

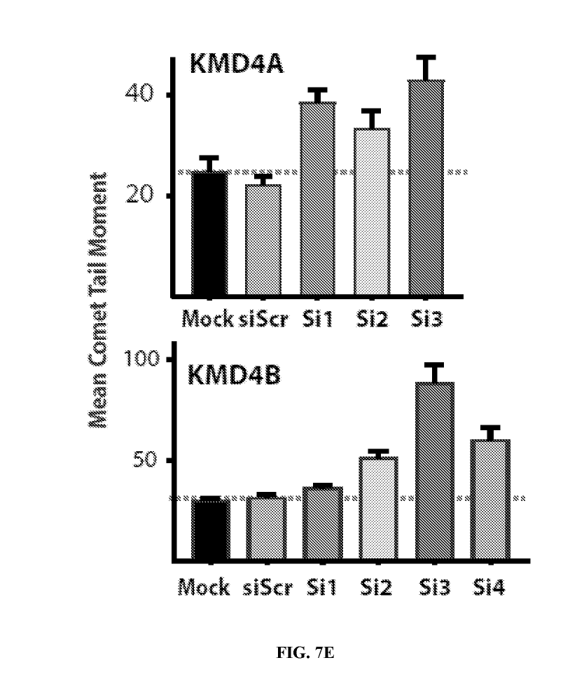

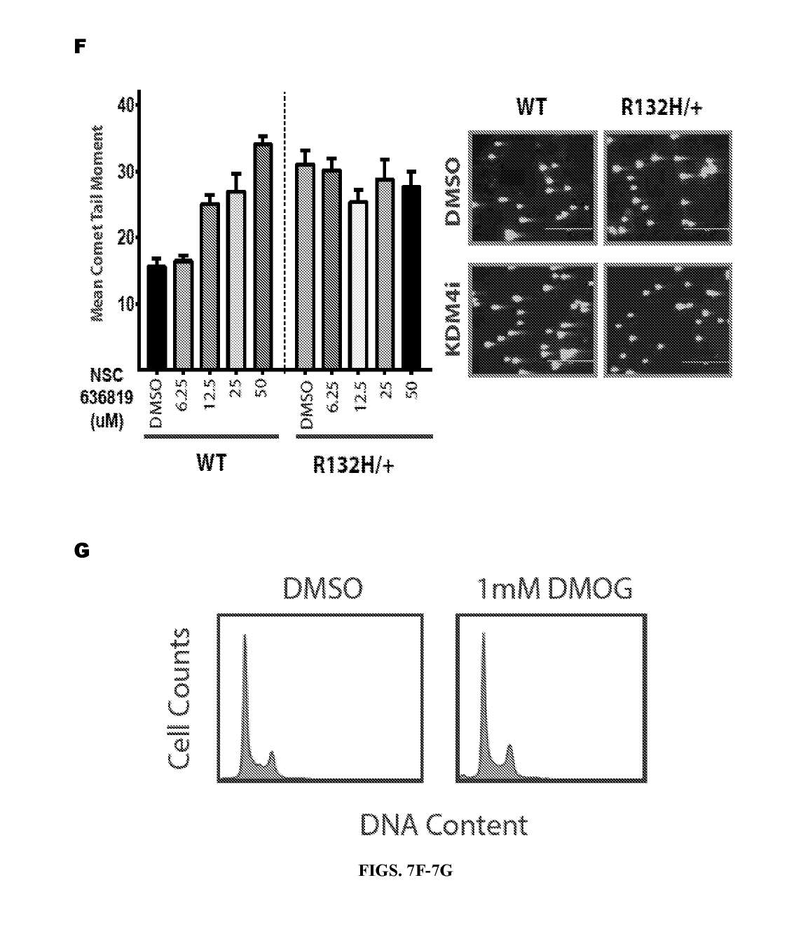

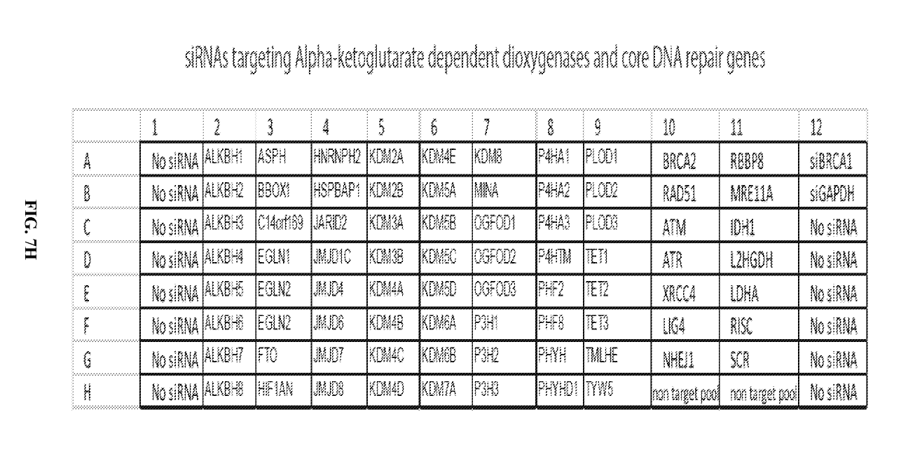

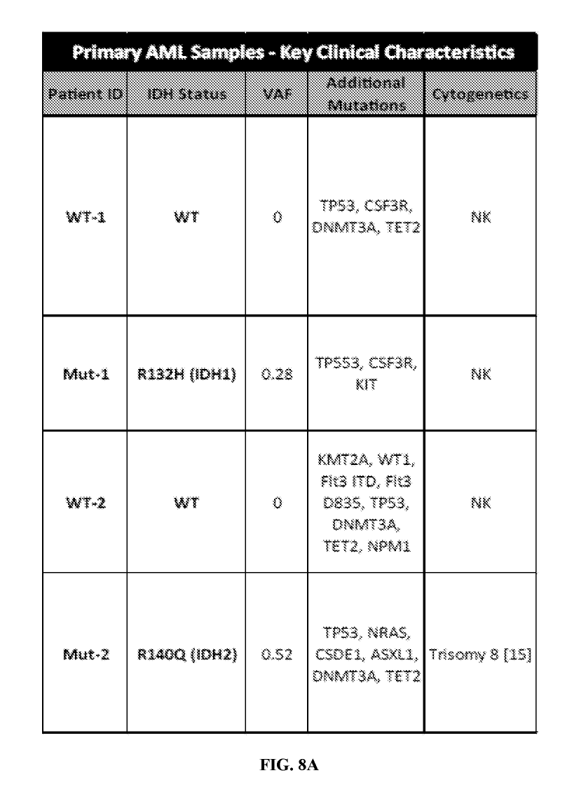

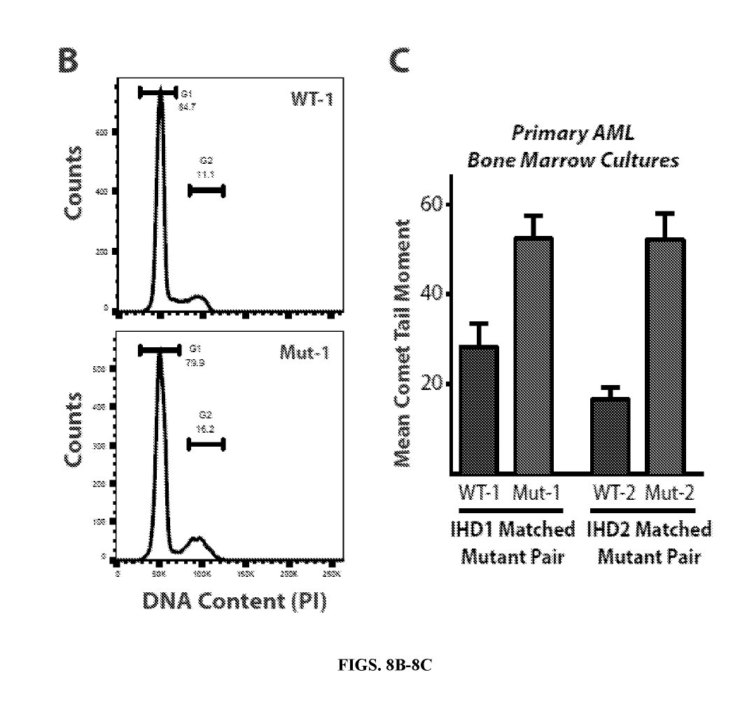

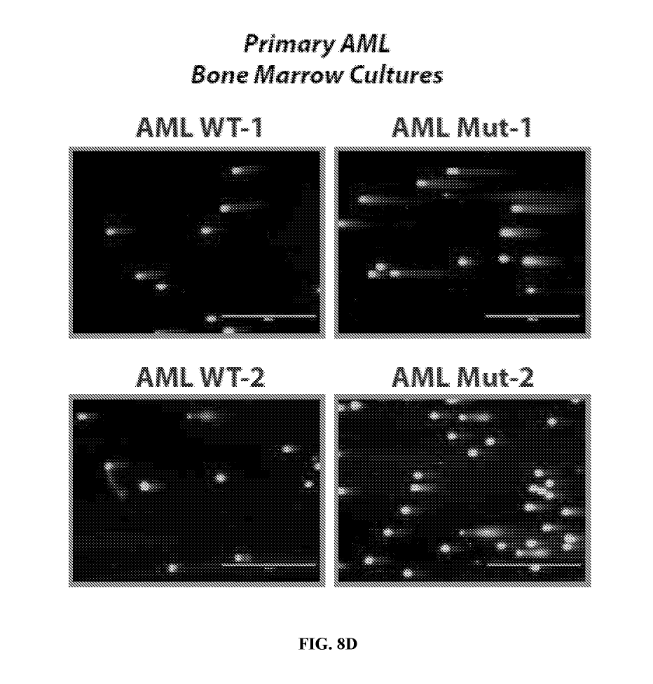

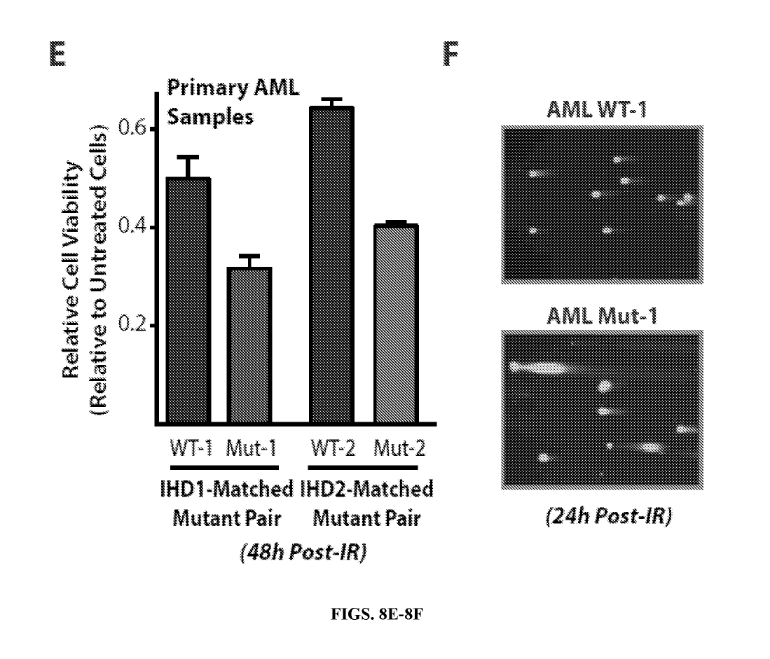

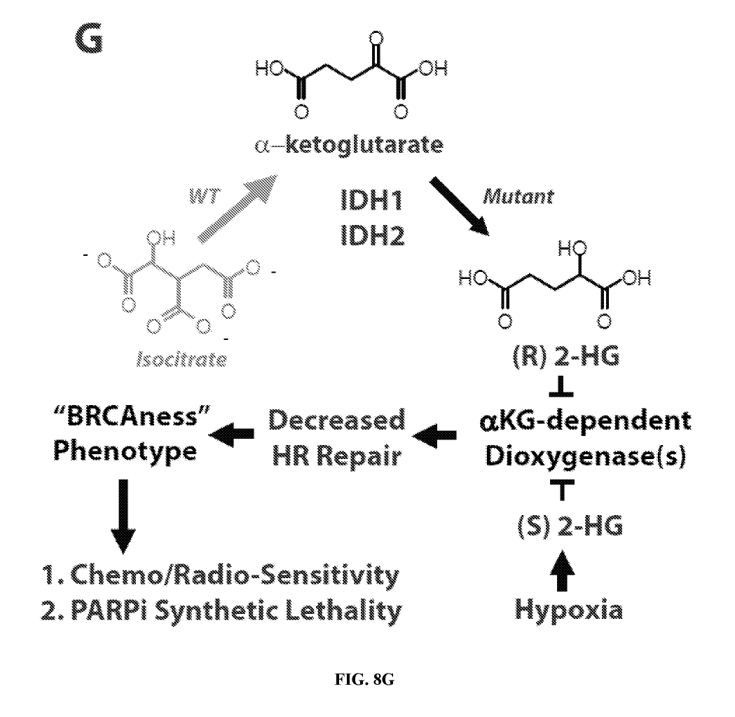

Both the (R) and (S) forms of 2HG are believed to exert their effects primarily via direct inhibition of αKG-dependent dioxygenases. The possibility of inducing a similar HR defect via treatment with a known inhibitor of these proteins, dimethyloxalylglycine (DMOG) (Baader et al., 1994, The Biochemical journal 300 (Pt 2), 525-530) was tested. DMOG is a structural analog of αKG in which the —CH₂-moiety has been replaced by an —NH—, and it acts as a competitive inhibitor of αKG-dependent dioxygenases (Baader et al., 1994, The Biochemical journal 300 (Pt 2), 525-530; Xu et al., 2011, Cancer cell 19, 17-30). As shown in FIG. 3K, treatment of IDH1-WT U2OS DR-GFP cells with DMOG resulted in a dose dependent increase in baseline DSBs (FIG. 3J), which correlated with dose dependent HR suppression (FIG. 3J). No major changes in cell cycle phase distribution were observed after treatment with DMOG (or with the octyl ester forms of 2HG or αKG), and thus these effects cannot be explained by a confounding G1/G0-arrest phenotype (Supplementary FIG. 6D). A focused siRNA screen targeting all major αKG-dependent dioxygenases was conducted in order to identify the protein(s) involved in the 2HG-induced HR suppression phenotype. U2OS DR-GFP cell line optimized for use in 96- and 384-well microplate screening campaigns was utilized. The genes targeted with pooled siRNAs (4 siRNAs per gene) are shown in FIG. 7H. This screen pointed to a handful of αKG-dependent dioxygenase genes, the knock down of which yielded Z-scores (for the HR suppression phenotype) that clustered with the effects of exposure to (R)-2HG, (S)-2HG and DMOG (FIG. 3L). Deconvolution of the corresponding siRNA pools narrowed the candidate list to two key aKG-dependent dioxygenase genes, KDM4A and KDM4B, based on the criteria that three or more siRNAs targeting these two genes led to a substantial reduction in HR activity compared to scrambled control siRNAs (FIG. 3M). Each of the active siRNAs also induced a substantial increase in baseline elevated DSBs in HCT116 IDH1-WT cells, which served as an orthogonal validation of specificity (FIG. 3N). A test to determine whether a similar phenotype was inducible in the same IDH1-WT cells with a highly selective small molecule inhibitor of KDM4A/KDM4B, NSC-636819 was conducted. As shown in FIG. 3O, the treatment of HCT116 IDH-WT cells with this drug induced a dose-dependent increase in elevated DSBs, which approached the levels seen in untreated HCT116 IDH-mutant cells. This phenotype was observed at doses ranging from 12.5-50 μM, which is within the reported range for the activity of this drug in cell culture studies (Wang et al., 2016, Cell Rep 16, 3016-3027; Chu et al., 2014, J Med Chem 57, 5975-5985). Importantly, NSC-636819 drug treatment in HCT116 IDH-mutant cells did not further increase comet tail moments, suggesting an epistatic interaction with the mutant IDH1 gene (FIG. 3O). Importantly, both KDM4A and KDM4B are histone lysine demethylases that have already been shown to play roles in the orchestration of DSB repair and recruitment of repair factors to sites of DNA damage (Mallette et al., 2012, EMBO J 31, 1865-1878; Young et al., 2013, J Biol Chem 288, 21376-21388), providing the mechanistic basis to link 2HG inhibition of these dioxygenases to attenuation of DNA repair.

### Example 3

### Patient-Derived AML Cells Show IDH1 Mutation-Associated and 2HG-Mediated HR Suppression and PARP Inhibitor Sensitivity

As an additional test of clinical relevance, a test to determine whether a mutant IDH1/2-dependent DSB repair defect could be detected in primary bone marrow cultures derived from AML patients with tumors harboring IDH1 and IDH2 gene mutations was conducted. The clinical characteristics of four specimens are shown in FIG. 6A. These cultures were successfully maintained and expanded for several passages, and this was confirmed through a log-phase proliferation (representative cell cycle plots are shown in FIG. 6B for two samples). Similar to the results obtained with the primary glioma cell lines, IDH-mutation associated elevations in DSBs were detected in a matched IDH1-WT and -mutant pair of primary samples, and similar results were obtained with a matched IDH2-WT and -mutant pair (FIG. 6C; representative comet images shown in FIG. 6D). An increased radiosensitivity was also detected in both the IDH1- and IDH2-mutant AML cells compared to their WT counterparts (FIG. 6E), which correlated with prolonged DSBs 24 h post-IR (FIG. 6F).

### Example 4

In summary, the present invention provides herein a novel and unexpected finding that neomorphic IDH1 mutations induce an HR defect that renders tumor cells sensitive to PARP inhibition in vitro and in vivo. This phenotype can be entirely recapitulated by 2-HG exposure, and it is correlated with reduced mRNA levels of several key HR genes. Suppressed HR gene expression in IDH1-mutant tumors was confirmed in two independent glioma cohorts, and these findings were validated using isogenic, human IDH1 WT and mutant cell lines in vitro.

A proposed mechanism of action is summarized in FIG. 21. Both IDH1-induced (R)-2-HG and hypoxia-induced (S)-2-HG suppress α-KG-dependent dioxygenases, leading to profound epigenetic reprogramming in cells (Losman et al., Genes Dev 27, 836-852 (2013); Intlekofer et al., Cell metabolism 22, 304-311 (2015)). Several HR pathway genes, including those reported herein, are epigenetically suppressed by hypoxia via shared gene promoter elements, leading to genetic instability and a BRCAness phenotype (Lu et al., Molecular and cellular biology 31, 3339-3350 (2011); Bindra et al., Cancer biology & therapy 5, 1400-1407 (2006); Scanlon et al., Molecular cancer research: MCR 12, 1016-1028 (2014)). The findings reported herein provide a novel link between hypoxia and IDH1 mutations as a common “hit-and-run” mechanism for genetic instability and tumor progression, which can be therapeutically exploited. Clinical trials are now testing synthetic lethal targeting of hypoxia-induced HR suppression by combining cediranib, an angiogenesis inhibitor that induces transient hypoxia, with PARP inhibitors as a novel therapeutic strategy (e.g., NCT01116648), further highlighting the clinical relevance of the present results. IDH1-mutant gliomas are known to be chemo- and radiosensitive, although the mechanisms underlying this enhanced sensitivity have been elusive (Tran et al., Neuro-oncology 16, 414-420 (2014)). The finding that IDH1 mutations induce “BRCAness” provides a basis for this molecular interaction. As PARP inhibitors are currently in clinical trials, and one is FDA-approved for HR-deficient cancers (olaparib), the data assessed herein suggest an urgent need to test these agents in IDH1-mutant gliomas and other tumors with upregulated 2-HG production. Small molecule inhibition of oncogenic kinases is a pillar of precision medicine in modern oncology (Gross et al., The Journal of clinical investigation 125, 1780-1789 (2015)), and many have extrapolated this approach to treat IDH1-mutant tumors (i.e., by direct inhibition of the mutant protein) (Dang et al., Annals of oncology: official journal of the European Society for Medical Oncology/ESMO 27, 599-608 (2016)). The present study raises caution regarding therapies that could revert a phenotype associated with a more favorable prognosis, and importantly, diminish an acquired vulnerability that could potentially be exploited for a therapeutic gain.

The disclosures of each and every patent, patent application, and publication cited herein are hereby incorporated herein by reference in their entirety.

While the present invention has been disclosed with reference to specific embodiments, it is apparent that other embodiments and variations of the present invention may be devised by others skilled in the art without departing from the true spirit and scope of the invention. The appended claims are intended to be construed to include all such embodiments and equivalent variations.

What is claimed is:

1. A method of treating or preventing a cancer in a mammalian subject in need thereof, the method comprising administering to the subject a therapeutically effective amount of at least one compound selected from the group consisting of a DNA repair inhibitor, a DNA strand break repair inhibitor, and a homologous recombination (HR) repair inhibitor, wherein cells in the cancer comprise an isocitrate dehydrogenase (IDH) mutation.

2. The method of claim 1, wherein the IDH mutation comprises a mutation in IDH1 or IDH2.

3. The method of claim 1, wherein the at least one compound comprises at least one poly(ADP-ribose) polymerase (PARP) inhibitor selected from the group consisting of olaparib, Iniparib, Niraparib, Veliparib, Rucaparib, 3-aminobenzamide and BMN-673 (Talazoparib), or at least one alpha-ketoglutarate-dependent dioxygenase A or B (KDM4A or KDM4B) inhibitor selected from the group consisting of DMOG, NSC 636819, PK 118 310, NCGC 00247751, NCGC 00244536, NCGC 00247743, IXO1, Disulfiram and JIB04.

4. The method of claim 1, wherein the subject is further administered at least one antitumor agent.

5. The method of claim 4, wherein the antitumor agent is selected from the group consisting of a topoisomerase inhibitor, an alkylating agent, nitrosoureas, an antimetabolite, an antitumor antibiotic, an antimicrotubule agent, a hormonal agent, a DNA strand break inducing agent, an epidermal growth factor (EGF) receptor inhibitor, an anti-EGF receptor antibody, an AKT inhibitor, an mTOR inhibitor, a CDK inhibitor, a tyrosine kinase receptor (TKR) inhibitor, a serine/threonine kinase inhibitor, a phosphatidyl inositol 3-kinase-like (PIKK) protein kinase inhibitor, a DNA dependent protein kinase (DNA-PK) inhibitor, an Ataxia Telangiectasia Mutated (ATM) inhibitor, an Ataxia Telangiectasia and Rad3 Related (ATR) inhibitor, a ribonucleotide reductase inhibitor, and an immune checkpoint inhibitor.

6. The method of claim 4, wherein treatment of the subject with the at least one compound and at least one antitumor agent is synergistic.

7. The method of claim 4, wherein the at least one compound and at least one antitumor agent are co-administered to the subject.

8. The method of claim 7, wherein the at least one compound and at least one antitumor agent are coformulated for administration to the subject.

9. The method of claim 1, wherein the subject is further administered radiation therapy.

10. The method of claim 9, wherein treatment of the subject with at least one compound and the radiation therapy is synergistic.

11. The method of claim 1, wherein the at least one compound is administered to the subject by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, pleural, peritoneal, subcutaneous, epidural, otic, intraocular, and topical.

12. The method of claim 1, wherein the cancer comprises at least one selected from the group consisting of brain head and neck cancer, glioma, meningioma, glioblastoma multiforme, lymphoma, leukemia, acute myeloid leukemia (AML), cholangiocarcinoma, multiple myeloma and neuroblastoma.

13. The method of claim 12, wherein the cancer comprises glioma, acute myelogenous leukemia or cholangiocarcinoma.

14. The method of claim 1, wherein the mammal is a human.

15. A method of treating a cancer in a mammalian subject in need thereof, the method comprising administering to the subject a therapeutically effective amount of at least one compound selected from the group consisting of 2-hydroxyglutarate (2-HG), a derivative of 2-HG, any variant and any mixtures thereof

16. The method of claim 15, wherein the cancer does not comprise an isocitrate dehydrogenase (IDH) mutation.

17. The method of claim 15, wherein the at least one compound induces a defect in DNA repair, DNA strand break repair or a homologous recombination (HR) of the cancer cells in the subject.

18. The method of claim 15, wherein the subject is further administered at least one antitumor agent and one poly(ADP-ribose) polymerase (PARP) inhibitor.

19. The method of claim 18, wherein the PARP inhibitor is selected from the group consisting of olaparib, Iniparib, Niraparib, Veliparib, Rucaparib, 3-aminobenzamide and BMN-673 (Talazoparib).

20. The method of claim 18, wherein the antitumor agent is selected from the group consisting of a topoisomerase inhibitor, an alkylating agent, nitrosoureas, an antimetabolite, an antitumor antibiotic, an antimicrotubule agent, a hormonal agent, a DNA strand break inducing agent, an epidermal growth factor (EGF) receptor inhibitor, an anti-EGF receptor antibody, an AKT inhibitor, an mTOR inhibitor, a CDK inhibitor, a tyrosine kinase receptor (TKR) inhibitor, a serine/threonine kinase inhibitor, a PIKK protein kinase inhibitor, a DNA-PK inhibitor, an ATM inhibitor, an ATR inhibitor, a ribonucleotide reductase inhibitor, and an immune checkpoint inhibitor.

21. The method of claim 18, wherein treatment of the subject with the at least one compound, the at least one antitumor agent and the PARP inhibitor is synergistic.

22. The method of claim 18, wherein the at least one compound, the at least one additional antitumor agent and the PARP inhibitor are co-administered to the subject.

23. The method of claim 18, wherein the at least one compound, the at least one additional antitumor agent and the PARP inhibitor are coformulated for administration to the subj ect.

24. The method of claim 15, further comprising administration of a PARP inhibitor and a radiation therapy to the subject.

25. The method of claim 24, wherein treatment of the subject with the at least one compound and the PARP inhibitor and radiation therapy is synergistic.

26. The method of claim 15, wherein the at least one compound is administered to the subject by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, pleural, peritoneal, subcutaneous, epidural, otic, intraocular, and topical.

27. The method of claim 15, wherein the cancer comprises at least one selected from the group consisting of breast cancer, prostate cancer, ovarian cancer, cervical cancer, skin cancer, pancreatic cancer, colorectal cancer, renal cancer, liver cancer, brain cancer, glioma, meningioma, glioblastoma multiforme, melanoma, lymphoma, leukemia, acute myeloid leukemia (AML), cholangiocarcinoma, lung cancer, endometrial cancer, head and neck cancer, sarcoma, multiple myeloma and neuroblastoma.

28. The method of claim 15, wherein the cancer comprises cells defective in at least one protein selected from the group consisting of BRCA1, BRCA2, PTEN, ATM, ATR, PALB2, FANCD2, RAD50, RAD51, other component of the homology dependent DNA repair pathway or the non-homologous end joining pathway or other component that mediate or regulate DNA repair.

29. The method of claim 15, wherein the derivative of 2-HG is (2R)-octyl-α-hydroxyglutarate (octyl-(R)-2HG).

30. The method of claim 15, wherein the mammal is a human.

31. A pharmaceutical composition comprising an anti-tumor effective amount of at least one compound selected from the group consisting of 2-HG, a derivative of 2-HG, any variant thereof, and any mixtures thereof.


SEQUENCE LISTING (4 sequences)

SEQ ID NO: 1 - DNA, 39 bp, Artificial Sequence
  Sequence for CRISPR/Cas-9
aggtcgtcatgcttatggggatcaagtaagtatgttggc

SEQ ID NO: 2 - DNA, 39 bp, Artificial Sequence
  Sequence for CRISPR/Cas-9
aggtcatcatgcttatggtgatcaagtaagtatgttggc

SEQ ID NO: 3 - DNA, 24 bp, Homo sapiens
ggtcatcatgcttatggtgatcaa

SEQ ID NO: 4 - DNA, 24 bp, Homo sapiens
ggtcgtcatgcttatggggatcaa